(19) United States
(12) Patent Application Publication
(10) Pub. No.: US 2026/0066044 A1
(43) Pub. Date: Mar. 5, 2026

(54) SYSTEM AND METHOD FOR PREDICTION OF UNINTENDED GENE EXPRESSION IMPACT IN A FERMENTATION PROCESS

(71) Applicant: Accenture Global Solutions Limited, Dublin (IE)

(72) Inventors: Christophe GUERET, Dublin (IE); Maziyar BARAN POUYAN, Petaluma, CA (US); Qurrat UL AIN, Dublin (IE)

(21) Appl. No.: 18/819,768

(22) Filed: Aug. 29, 2024

(51) Int. Cl. G16B 25/10; C12Q 1/6806; C12Q 1/6809

(52) U.S. Cl. CPC G16B 25/10; C12Q 1/6806; C12Q 1/6809

(57) ABSTRACT

A method includes (i) selecting a gene of a yeast to interact with one or more compounds for a mixture; (ii) creating a plurality of gene editions based on the gene; (iii) simulating expression of the plurality of gene editions to determine impact on the gene and a plurality of other genes; (iv) selecting a plurality of designated gene editions that satisfy at least one predetermined criteria; (v) simulating a reaction with the mixture and the yeast having individual ones of the plurality of designated gene editions; (vi) determining an expected final composition after a plurality of the simulated reactions; (vii) correlating data on a plurality of attributes of the expected final compositions to the desired final composition profile; and (viii) selecting and providing one or more expected final compositions and related gene edition data that most closely match the desired composition profile.

### TECHNICAL FIELD

The field of the disclosure relates generally to analytical pipelines predicting presence of components in a fermentation process, and in particular, to a prediction model for determining an impact of a particular gene editing process that is typically applied to increase yield of a target compound.

### REFERENCE TO THE SEQUENCE LISTING

The Sequence Listing submitted 29 Aug. 2024 as an XML file named “126401-814315-SQL”, created on 29 Aug. 2024 and having a size of 19.2 kilobytes is hereby incorporated by reference pursuant to 37 C.F.R. § 1.52(e)(5).

### BACKGROUND

Yeasts are mono-cellular organisms that work as micro-factories for producing proteins and other compounds. Yeasts are generally known for their role in brewing alcohol such as, wine, beer, etc., but are also used in the field of synthetic biology for production of specific proteins such as, casein used to produce “cow-less” milk, via a fermentation process. Irrespective of the end goal of the fermentation process, the value, quality, or truth of the fermentation process is determined based on experimentations or results of the experimentations. Generally, the yeast has to be given some days in a wet lab experiment to digest input compounds and produce the output, which is a lengthy time-consuming process. Further, at the end of the wet lab experiment, the quality or output of the fermentation process may not be according to the desired expectations, which would require repetitions of the wet lab experiments to identify the correct recipe for the fermentation process. Further, based upon how the fermentation process is performed, different compounds are generated affecting quality or flavor of a product generated using the fermentation process output. Additionally, when gene edited cells are used during the fermentation process, based upon which gene is edited, the quality or flavor of the product generated using the fermentation process output may differ substantially.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY

In one aspect, a method of selecting an engineered organism with a desired protein expression is disclosed. The method includes (i) receiving a request for a desired composition profile produced from a mixture; (ii) analyzing the mixture to determine a respective quantity of a plurality of different compounds in the mixture; (iii) selecting a gene of a yeast to interact with one or more of the compounds; (iv) creating a plurality of gene editions based on the gene; (v) simulating expression of the plurality of gene editions to determine impact on the gene and a plurality of other genes; (vi) selecting, from the plurality of gene editions, a plurality of designated gene editions that satisfy at least one predetermined criteria; (vii) simulating a reaction with the mixture and the yeast having individual ones of the plurality of designated gene editions, wherein the simulating includes a plurality of hours, the mixture, and the yeast having the individual ones of the plurality of designated gene editions; (viii) determining an expected final composition after a plurality of the simulated reactions; (ix) correlating data on a plurality of attributes of the expected final compositions to the desired final composition profile; (x) selecting one or more expected final compositions that most closely match the desired composition profile; and (xi) providing related gene edition data that produced the selected one or more expected final compositions.

In some examples, the mixture is feedstock for a synthetic biology production.

In some examples, the plurality of attributes includes one or more of hoppy, fruity, sulfury, bitter, floral, citrus, green, spicy, and/or sweet.

In some examples, the stimulating the expression of the plurality of gene editions includes inferring, based upon a tree-based machine learning algorithm, an initial metabolite precursor of n number of initial yeast cells.

In some examples, the tree-based machine learning algorithm includes a random forest regression model trained to predict an expression level of the gene based on corresponding expression levels of all other genes in the data set.

In some examples, the tree-based machine learning algorithm is constructed by recursively splitting data into smaller subsets based on expression levels of a randomly selected subset of genes until a predetermined criteria is satisfied.

In some examples, analyzing gene interactions to determine a combined effect including one or more of a synergistic interaction, complementary interaction, and/or modifier interaction.

In some examples, the method further includes executing a regularized linear model to determine influence of perturbations on gene expression. The regularized linear model predicts, based on at least one combined effect of guide molecules and/or initial regulatory network granulin precursors, expression levels of the gene and the all other genes.

In some examples, the method further includes validating the selection of the engineered organism, wherein the validating includes measuring reaction data.

In some examples, the method further includes validating the selection of the engineered organism includes measuring the expression of one or more genes of the engineered organism in a fermenting liquid or a fermented product.

In some examples, the method further includes repeating the measuring the expression of one or more genes of the engineered organism in a fermenting liquid.

In some examples, the measuring the expression of one or more genes includes using high-density expression array, DNA microarray, polymerase chain reaction (PCR), reverse transcriptase PCR (RT-PCR), real-time quantitative reverse transcription PCR (qRT-PCR), serial analysis of gene expression (SAGE), spotted cDNA arrays, GeneChip, spotted oligo arrays, bead arrays, RNA Seq, tiling array, northern blotting, hybridization microarray, in situ hybridization, whole-exome sequencing, whole-genome sequencing, liquid biopsy, next-generation sequencing, or any combination thereof.

In some examples, the measuring the reaction data includes analyzing the chemical composition of fermenting liquid or fermented product.

In some examples, the measuring the reaction data includes analyzing the flavor composition of fermenting liquid or fermented product.

In some examples, the validating the selection of the engineered organism includes building a network of gene regulation data.

In some examples, the method further includes identifying gene regulation data associated with a desired flavor profile.

In some examples, the method further includes predicting the flavor profile based on chemical composition data.

In some examples, the desired flavor profile includes Ethyl Acetate (EA), Ethanol, 1-Propanol (1P), 1-Propanol, 2-methyl-(1P2M), 1-Butanol, 3-Methyl-(1B3M), Ethyl Ester Octanoic Acid (OCE), 2-Oxo-Propanoic Acid (2PA), 2-Phenylethyl Acetate (2-PEAC), Hexanoic acid (HA), Phenylethyl Alcohol (PA), 1,2-Benzisothiazole (12B), Octanoic Acid (OA), n-Decanoic acid (nDC), 2,4-Di-tert-butylphenol (24DTB), 9-Decenoic Acid (9DC), Acetaldehyde, acetoxy-3-methoxycinnamaldehyde (4A3M), 1-Butanol, 3-methyl-, acetate (IA), 1-Butanol, 2-methyl-, acetate (AAA), Decanoic acid, ethyl ester (DCE), Hexanoic acid, ethyl ester (HCE), Oxalic Acid, Cyclobutyl Hexadecyl Ester (OCHE), Butanoic acid, butyl ester (BB), 2-Ethyl-Heptanoic Acid (2EHC), 1-Heptanol, 2-Propyn-1-ol (2PP), or any combination thereof.

In some examples, the method further includes introducing one or more gene editions into a native or wild-type organism to create an engineered organism.

In some examples, the method further includes introducing one or more gene editions using a gene editing system.

In some examples, the gene editing system comprises using zinc-finger nucleases (ZFNs), transcription activator-like effector nucleases (TALENs), meganucleases, or clustered regularly interspaced short palindromic repeats (CRISPR).

In some examples, CRISPR comprises CRISPR/Cas9.

In some examples, the gene editions modulate the expression level of one or more genes.

In some examples, the gene editions modulate the expression level of one or more genes involved in one or more biological processes.

In some examples, the modulate includes increasing the expression level of one or more genes, or wherein modulating comprises decreasing the expression of one or more genes.

In some examples, the modulate comprises increasing the expression level of one or more genes and decreasing the expression of one or more genes.

In some examples, the one or more biological processes include peptide biosynthesis, cellular amide metabolism, amide biosynthesis, peptide metabolism, organonitrogen compound biosynthesis, biosynthesis, macromolecule biosynthesis, organic substance biosynthesis, cellular macromolecule biosynthesis, cellular biosynthesis, cellular protein metabolism, gene expression, cellular nitrogen compound biosynthesis, protein metabolism, organonitrogen compound metabolism, response to oxidative stress, anion transport, cellular macromolecule metabolism, aspartate family amino acid metabolism, carboxylic acid biosynthesis, organic acid biosynthesis, branched-chain amino acid metabolism, branched-chain amino acid biosynthesis, sulfur amino acid metabolism, fatty acid biosynthesis, monocarboxylic acid biosynthesis, small molecule biosynthesis, fatty acid metabolism, sulfur amino acid biosynthesis, energy derivation by oxidation of organic compounds, or any combinations thereof.

In some examples, the one or more gene editions affect ADH1-ADH7, (alcohol dehydrogenase); ALD (alanine dehydrogenase); ARO10 (phenylpyruvate decarboxylase); BAT1 (branched-chain-amino-acid); transaminase); BAT2 (branched-chain-amino-acid transaminase); CHA1 (1-serine/1-threonine ammonia-lyase); EHT1 (medium-chain fatty acid ethyl ester synthase/esterase), FAA4 (fatty acid activation 4); HOR7; ILV3 (dihydroxy-acid dehydratase); ILV5 (ketol-acid reductoisomerase); ILV6 (acetolactate synthase regulatory subunit); IMA1 (isomaltase); IRA1 (GTPase-activating protein); IRC7; LEU1 (3-isopropylmalate dehydratase); LEU2 (3-isopropylmalate dehydrogenase); LEU4 (2-isopropylmalate synthase); LEU9 (2-isopropylmalate synthase); QCR7 (ubiquinol-cytochrome C oxidoreductase); OLE1 (oleic acid requiring); PDC (pyruvate decarboxylase); PUT1 (proline utilization); SFA1 (bifunctional alcohol dehydrogenase), SSD1, THI3 (branched-chain-2-oxoacid decarboxylase); TPS2 (Trehalose-6-Phosphate Synthase/phosphatase); or any combination thereof.

In some examples, the engineered organism is yeast.

In some examples, the engineered organism is Saccharomyces cerevisiae or Saccharomyces pastorianus.

In another aspect, a system for selecting an engineered organism with a desired protein expression is disclosed. The system includes at least one memory storing instructions, and at least one processor communicatively coupled with the at least one memory. The at least one processor is configured to perform operations including (i) receiving a request for a desired composition profile produced from a mixture; (ii) analyzing the mixture to determine a respective quantity of a plurality of different compounds in the mixture; (iii) selecting a gene of a yeast to interact with one or more of the compounds; (iv) creating a plurality of gene editions based on the gene; (v) simulating expression of the plurality of gene editions to determine impact on the gene and a plurality of other genes; (vi) selecting, from the plurality of gene editions, a plurality of designated gene editions that satisfy at least one predetermined criteria; (vii) simulating a reaction with the mixture and the yeast having individual ones of the plurality of designated gene editions, wherein the simulating includes a plurality of hours, the mixture, and the yeast having the individual ones of the plurality of designated gene editions; (viii) determining an expected final composition after a plurality of the simulated reactions; (ix) correlating data on a plurality of attributes of the expected final compositions to the desired final composition profile; (x) selecting one or more expected final compositions that most closely match the desired composition profile; and (xi) providing related gene edition data that produced the selected one or more expected final compositions.

In another aspect, a non-transitory computer-readable media including instructions stored thereon is disclosed. The instruction, which, when executed by at least one processor of at least one computing device for selecting an engineered organism with a desired protein expression, cause the at least one computing device to perform operations including (i) receiving a request for a desired composition profile produced from a mixture; (ii) analyzing the mixture to determine a respective quantity of a plurality of different compounds in the mixture; (iii) selecting a gene of a yeast to interact with one or more of the compounds; (iv) creating a plurality of gene editions based on the gene; (v) simulating expression of the plurality of gene editions to determine impact on the gene and a plurality of other genes; (vi) selecting, from the plurality of gene editions, a plurality of designated gene editions that satisfy at least one predetermined criteria; (vii) simulating a reaction with the mixture and the yeast having individual ones of the plurality of designated gene editions, wherein the simulating includes a plurality of hours, the mixture, and the yeast having the individual ones of the plurality of designated gene editions; (viii) determining an expected final composition after a plurality of the simulated reactions; (ix) correlating data on a plurality of attributes of the expected final compositions to the desired final composition profile; (x) selecting one or more expected final compositions that most closely match the desired composition profile; and (xi) providing related gene edition data that produced the selected one or more expected final compositions.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is an example of a known beer fermentation process for evaluation of different yeast strains (e.g., different variants of the same yeast organism.

FIG. 2A is a table of volatile organic compounds identified in twelve different beer samples.

FIG. 2B is a table showing a qualitative evaluation of twelve different beer samples.

FIG. 3 is an example pipeline to predict an outcome of in-silico fermentation process.

FIG. 4 is an example diagram illustrating an overview of use-cases derived based upon results shown in tables of FIG. 2A and FIG. 2B, respectively, for twelve different yeast strains.

FIG. 5 is an example flow-diagram of gene expression analysis for one use-case shown in FIG. 4.

FIG. 6 is an example flow-diagram of flavor prediction for another use-case shown in FIG. 4.

FIG. 7 is an example flavor profile generated using compound fingerprinting shown in FIG. 6.

FIG. 8 is an example flavor profile generated using the PCM modeling.

FIG. 9 is an example flow-chart of steps or operations involved in up-regulation of the IRC7 gene-expression.

FIG. 10 is a block diagram of an example computing system.

FIG. 11 is an example flow-chart of method operations for selecting an engineered organism with a desired protein expression.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing.

Some structural or method features may be shown in specific arrangements and/or orderings in the drawings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments, and, in some embodiments, it may not be included or may be combined with other features.

### DETAILED DESCRIPTION

The following detailed description and examples set forth preferred materials, components, and procedures used in accordance with the present disclosure. This description and these examples, however, are provided by way of illustration only, and nothing therein shall be deemed to be a limitation upon the overall scope of the present disclosure.

One or more of the following terms may be used in the disclosure, and their definition is provided below.

Yeast: Yeasts are mono-cellular organisms classified in the kingdom of fungus. Yeasts are used for millennia for fermentation processes producing wine, beer, and other beverages. More recently, by applying principles of synthetic biology, yeasts are used to produce a wide range of compounds including, but not limited to, casein, fragrances, pigments, biofuels, pharmaceuticals, and/or additives, etc.

Yeast strain: A yeast strain is a specific variant of a yeast organism. For example, LalBrew Abbaye and LalBrew London as two strains of the same yeast organism Saccharomyces cerevisiae producing very different beers due to the difference or high variability in their genetic material.

Shelf life: The shelf life is a measure of time corresponding to the duration for a product to remain stable. Generally, the shelf life is encountered as a logic behind the best before date found on products. Different yeast strains or fermentation processes impact the shelf life differently, and, therefore, the commercial value of the product.

In an aspect, “gene regulatory network” or GRN refers to a gene regulatory network (GRN) is a model of the different type of gene interaction. The main interactions are up-regulation and down-regulation. The GRN of a particular organism is a tool that can be used to predict a knock-on effect when modifying the expression of a particular target gene.

Genes rarely act in isolation; instead, they interact with each other and make up gene regulatory networks to function as a whole. The study of this mechanism is crucial for understanding the properties and functions of genes, which help reveal the genetic architecture of complex traits and diseases. Although genetic experiments can be conducted to discover interactions among genes, this approach can be costly and time consuming. Alternatively, measurements of gene expression levels reveal gene expression patterns in a specific condition and can be exploited to infer gene regulatory networks. Various approaches have been proposed to infer gene regulatory networks using gene expression data, such as relevance networks, Bayesian networks, Gaussian graphical models, and many others.

The gene regulation network is a biochemical network formed by a group of genes, proteins, small molecules and mutual regulation and control effects among the genes, the proteins and the small molecules, and is a basic and important biological network. The biological control theory combining biology and control theory is an important component of the control theory, and the gene regulation network is an important branch of the biological control theory, which can skillfully apply the control, regulation and cooperation in the biological system to a multi-agent system. The Turing reaction-diffusion model is a classical and effective model for studying biological pattern formation, and has been greatly developed in recent decades.

In an aspect, the gene regulation network modeling is mainly based on the regulation relationship in the gene expression data reasoning network, and is expressed as a topological structure, and belongs to the research of reverse engineering by data mining. The construction of the gene regulation network firstly needs to determine a network model, and then selects a proper modeling algorithm according to the model. Classical network models include Boolean networks, associative networks, differential equations, and Bayesian networks.

For example, in an aspect, a Boolean network makes corresponding simplification to the gene state, and uses Boolean function to replace differential and derivative to describe the correlation between genes. The model has the defects of inaccuracy, the fact that the real gene regulation network topological structure cannot be accurately described only by describing and reflecting the interaction between genes by using a fixed logic rule, and the loss of important expression information is inevitably caused when gene data are discretized. Probabilistic Boolean Network (PBN), which is an extension of the conventional Boolean network, simultaneously quantifies the interaction relationship and sensitivity between genes to solve the uncertainty in the model selection process and improve the accuracy of the model.

In an aspect, associating the network is modeling of the association network is mainly realized by the association degree between gene expression data. The similarity between genes is usually calculated by using measures such as mutual information, Pearson correlation coefficient and the like, and if the similarity between the gene pairs is higher than a certain threshold value, the gene pairs are directly connected in the network. Button et al first calculates the degree of association between all pairs of genes using mutual information, and then sets a threshold value for the mutual information. Later, it was found that if the gene pairs have the same or similar regulatory mechanism, the association between the two genes is higher, especially the target genes of the same transcription factor or the genes on the same biological pathway. To reduce the false positive rate of the constructed network structure and obtain a regulation and control network close to a real topology, the influence of other genes is isolated when the association degree between the gene pairs is calculated.

In an aspect, a Bayesian Network (BN) approximately describes the complex probability distribution of the whole network structure through the product of local probabilities, belongs to a probability graph model, and expresses connecting edges among nodes as probability dependency existing among the nodes. The Dynamic Bayesian Network (DBN) is an extension of a static Bayesian network model, and a dynamic change network is formed by introducing time factors, so that the dynamic property of a stochastic system is more truly represented. The gene regulation network is a complex and continuous dynamic network system in nature, so that the DBN is often simplified during specific modeling, thereby reducing the computational complexity. The DBN overcomes the defect that static BN has directional acyclic, better describes the dynamic characteristics of a gene regulation network, and improves the prediction precision of a model.

Norbert uses a discretization method to preprocess gene expression data in order to learn a dynamic Bayesian network from gene perturbation type experimental data, provides a new data integration model by combining negative feedback and time delay factors of gene regulation, and uses a parallel algorithm to accelerate the construction of a gene regulation network.

Gene regulatory networks can be characterized using a system of structural equations, with each equation describing the causal effects of cis-eQTL and the regulatory effects of other genes on a given gene. Such a framework makes it feasible to take a genome-wide survey and to directly reveal interactions among genes. Application of structural equations in genetical genomics studies have been previously demonstrated. Two studies are applicable to constructing gene regulatory networks for a small number of genes. However, genetical genomics experiments usually collect whole-genome gene expressions for a very limited number of samples, therefore the number of genes is much larger than the sample size. For such consideration, another study proposed to apply the adaptive lasso to construct a sparse gene regulatory network. An additional approach instead proposed to maximize a penalized likelihood for constructing a sparse gene regulatory network.

Elucidating relationships between genes, and the products they encode, remains one of the central challenges in experimental and computational biology. A gene regulatory network (GRN) is a directed graph in which regulators of gene expression are connected to target gene nodes by interaction edges. Regulators of gene expression include transcription factors (TF) which can act as activators and repressors, RNA binding proteins, and regulatory RNAs. Identifying regulatory relationships between transcriptional regulators and their targets is essential for understanding biological phenomena ranging from cell growth and division to cell differentiation and development. Reconstruction of GRNs is required to understand how gene expression dysregulation contributes to cancer and complex heritable diseases.

Genome-scale methods provide an efficient means of identifying gene regulatory relationships. Efforts of the past two decades have resulted in the development of a variety of experimental and computational methods that leverage advances in technology and machine learning for constructing GRNs. This method takes as inputs gene expression data and sources of prior information, and outputs regulatory relationships between transcription factors and their target genes that explain the observed gene expression levels. Subsequent work has enhanced this approach by selecting regulators for each gene more effectively, incorporating orthogonal data types that can be used to generate constraints on network structure, and explicitly estimating latent biophysical parameters including transcription factor activity and mRNA decay rates.

Recent advances in sequencing technologies make it feasible to obtain both whole-genome genotype and gene expression for yeast, i.e., genetical genomics data. Combining genetics with gene expression reveals additional information on genetic structure and holds great promise for improving the accuracy of gene regulatory network inference. Numerous genetical genomics experiments, such as the Genotype-Tissue Expression (GTEx) project, have been conducted to collect genetical genomics data.

Much effort has been devoted to using genetical genomics data for genome-wide association (GWA) analysis of gene expression, i.e., expression quantitative trait loci (eQTL) mapping. Mapping of eQTL intends to elucidate variation of expression traits attributed to genomic variation, and to identify chromosomal loci (i.e., eQTL) of genetic polymorphisms associated to the expression of a gene under investigation. An eQTL located within the region of the gene under investigation is called a cis-eQTL, otherwise it is called a trans-eQTL. While the cis effects of a gene represent direct regulations, indirect regulations of trans-eQTL are likely caused by interactions among genes. These eQTL provide insight on the functional sequences of the gene expression, and thus an indirect interrogation of the functional landscape of gene regulations.

In an aspect, synthetic biology is processes related to applying engineering principles to biology. An International Genetically Engineered Machine (iGEM) is an organization defining building blocks and collecting use-cases. Some examples of building blocks are Polymerase Chain Reaction (PCR) machinery creating deoxyribonucleic acid (DNA) copies from a single DNA sample, and the gene editing clustered regularly interspaced short palindromic repeats (CRISPR) and CRISPR-associated protein 9 (CRISPR-Cas9) that enables copying and pasting gene across organisms with a higher precision.

In an aspect, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Guide RNAs can be crafted to target IRC7, for example, or any other target gene. See, e.g., Table 1, below.

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8-al, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes.

In an aspect, “dCas9” refers to enzymatically inactive form of Cas9, which can bind, but cannot cleave, DNA. In an aspect, a disclosed variant Cas9 can comprise VQR, EQR, or VRER.

In an aspect, “Protospacer Adjacent Motif” or “PAM” refers to a sequence adjacent to the target sequence that is necessary for Cas enzymes to bind target DNA.

A “protospacer sequence” refers to the target double stranded DNA and specifically to the portion of the target DNA (e.g., or target region in the genome) that is fully or substantially complementary (and hybridizes) to the spacer sequence of the CRISPR arrays. The protospacer sequence in a Type I system is directly flanked at the 3′ end by a PAM. A spacer is designed to be complementary to the protospacer.

In general, a gRNA (also referred to herein as “gRNA scaffold” interchangeably) can complex with a compatible nucleic acid-guided nuclease and can hybridize with a target sequence, thereby directing the nuclease to the target sequence. A subject nucleic acid-guided nuclease capable of complexing with a guide polynucleotide can be referred to as a nucleic acid-guided nuclease that is compatible with the gRNA. In addition, a gRNA capable of complexing with a nucleic acid-guided nuclease can be referred to as a guide polynucleotide or a guide nucleic acid that is compatible with the nucleic acid-guided nucleases.

A gRNA can include a scaffold sequence. In general, a “scaffold sequence” can include any sequence that has sufficient sequence to promote formation of a targetable nuclease complex, wherein the targetable nuclease complex includes, but is not limited to, a nucleic acid-guided nuclease and a guide polynucleotide can include a scaffold sequence and a guide sequence. Sufficient sequence within the scaffold sequence to promote formation of a targetable nuclease complex can include a degree of complementarity along the length of two sequence regions within the scaffold sequence, such as one or two sequence regions involved in forming a secondary structure. In an aspect, the one or two sequence regions are included or encoded on the same polynucleotide. In an aspect, the one or two sequence regions are included or encoded on separate polynucleotides. Optimal alignment can be determined by any suitable alignment algorithm, and can further account for secondary structures, such as self-complementarity within either the one or two sequence regions. In an aspect, the degree of complementarity between the one or two sequence regions along the length of the shorter of the two when optimally aligned can be about or more than about 25%, 30%, 40%, 50%, 60%, 70%, 80%, 90%, 95%, 97.5%, 99%, or higher. In an aspect, at least one of the two sequence regions can be about or more than about 5, 6, 7, 8, 9, 10, 11, 12, 13, 14, 15, 16, 17, 18, 19, 20, 25, 30, 40, 50, or more nucleotides in length.

A scaffold sequence of a subject guide polynucleotide can comprise a secondary structure. A secondary structure can comprise a pseudoknot region. In an aspect, binding kinetics of a guide polynucleotide to a nucleic acid-guided nuclease is determined in part by secondary structures within the scaffold sequence. In an aspect, binding kinetics of a guide polynucleotide to a nucleic acid-guided nuclease is determined in part by nucleic acid sequence with the scaffold sequence.

In an aspect, a disclosed method can comprise validating a CRISPR event. In an aspect, validation of a CRISPR event can be accomplished using methods and techniques known to the art (e.g., sequencing, northern blots, FISH, PCR, RNA-Seq, 3′ RACE, 5′ RACE, etc.).

In an aspect, precision fermentation (PF) is a process by which an organism is modified to produce a specific compound and then set to ferment. As an example, PF is used for production of insulin and Casein (to produce milk-less milk) and Albumin (to make egg-less egg white).

Multi-omics datasets: Multi-omics datasets correspond with a biological analysis approach in which datasets are related to or associated with multiple “omes” such as, the genome, proteome, transcriptome, epigenome, metabolome, and microbiome (i.e., a meta-genome and/or meta-transcriptome).

In an aspect, in-silico refers to simulations of wet lab experiments on a computing device using computer simulation software or one or more machine learning algorithms for prediction of an output as if the wet lab experiment is performed.

In an aspect, “short hairpin RNA” or “shRNA” refers to sequence usually encoded in a DNA vector that can be introduced into cells via plasmid transfection or viral transduction. As known to the art, shRNA molecules can be divided into two main categories based on their designs: simple stem-loop and microRNA-adapted shRNA. A simple stem-loop shRNA is often transcribed under the control of an RNA Polymerase III (Pol III) promoter. The 50-70 nucleotide transcript forms a stem-loop structure consisting of a 19 to 29 bp region of double-strand RNA (the stem) bridged by a region of predominantly single-strand RNA (the loop) and a dinucleotide 3′ overhang.

The simple stem-loop shRNA is transcribed in the nucleus and enters the RNAi pathway similar to a pre-microRNA. The longer (>250 nucleotide) microRNA-adapted shRNA is a design that more closely resembles native pri-microRNA molecules and consists of a shRNA stem structure which may include microRNA-like mismatches, bridged by a loop and flanked by 5′ and 3′ endogenous microRNA sequences. The microRNA-adapted shRNA, like the simple stem-loop hairpin, is also transcribed in the nucleus but is thought to enter the RNAi pathway earlier similar to an endogenous pri-microRNA.

In an aspect, a disclosed method can comprise modulating expression of one or more genes. In an aspect, a disclosed method can comprise modulating expression of one or more target genes.

In an aspect, the one or more genes or the one or more target genes are in a cell or tissue. In an aspect, the one or more genes or the one or more target genes are in a cell or tissue in a subject. In an aspect, a target gene can comprise ADH1-ADH7, (alcohol dehydrogenase); ALD (alanine dehydrogenase); ARO10 (phenylpyruvate decarboxylase); BAT1 (branched-chain-amino-acid); transaminase); BAT2 (branched-chain-amino-acid transaminase); CHA1 (1-serine/1-threonine ammonia-lyase); EHT1 (medium-chain fatty acid ethyl ester synthase/esterase), FAA4 (fatty acid activation 4); HOR7; ILV3 (dihydroxy-acid dehydratase); ILV5 (ketol-acid reductoisomerase); ILV6 (acetolactate synthase regulatory subunit); IMA1 (isomaltase); IRA1 (GTPase-activating protein); IRC7; LEU1 (3-isopropylmalate dehydratase); LEU2 (3-isopropylmalate dehydrogenase); LEU4 (2-isopropylmalate synthase); LEU9 (2-isopropylmalate synthase); QCR7 (ubiquinol-cytochrome C oxidoreductase); OLE1 (oleic acid requiring); PDC (pyruvate decarboxylase); PUT1 (proline utilization); SFA1 (bifunctional alcohol dehydrogenase), SSD1, THI3 (branched-chain-2-oxoacid decarboxylase); TPS2 (Trehalose-6-Phosphate Synthase/phosphatase); or any combination thereof.

In an aspect, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, then expression can include splicing of the mRNA in a eukaryotic cell.

For example, in an aspect, IRC7 encodes cysteine-S-conjugate beta-lyase. IRC7 enables growth on cysteine as nitrogen source; involved in the production of thiols. The IRC7 null mutant displays increased levels of spontaneous Rad52p foci. IRC7 has a length of 340 amino acids and has molecular weight of 36964.7 Da. IRC7 has a gene ID of 850616.

In an aspect, ADH4 encodes alcohol dehydrogenase isoenzyme type IV. ADH4 enables alcohol dehydrogenase (NAD+) activity. ADH4 is involved in amino acid catabolic process to alcohol via Ehrlich pathway and fermentation. ADH4 has a length of 382 amino acids and a molecular weight of 41133.5 Da. ADH4 has a gene ID of 852635.

In an aspect, “target molecule” refers to a molecule of interest, the amount or expression level of which is directly or indirectly influenced by the activity of a fusion protein comprising the protein of interest fused in-frame with maltose-dependent degradation determinants. In an aspect, the term “target molecule” can refer to, for example, enzymes, other proteins, peptides, amino acids, nucleic acids, lipids, carbohydrates, metabolites, and non-catabolic compounds.

In an aspect, “functionally disrupted” or “functional disruption” of a selected gene can refer to an alteration of the selected gene in such a way that the activity of the protein encoded by the selected gene in the host cell is reduced. In an aspect, “functional disruption” or “functional disruption” of a selected protein means that the protein is altered in such a way that the activity of the protein in the host cell is reduced. In an aspect, the activity of the selected protein encoded by the selected gene is abolished in the host cell. In an aspect, the activity of the selected protein encoded by the selected gene is reduced in a host cell. In an aspect, functional disruption of the selected gene can be achieved by deleting all or part of the gene, thereby eliminating or reducing gene expression, or eliminating or reducing the activity of the gene product. In an aspect, functional disruption of the selected gene may also be achieved by mutating the regulatory elements of the gene, for example the promoter of the gene, thereby eliminating or reducing expression, or by mutating the coding sequence of the gene, such that the activity of the gene product is eliminated or reduced. In an aspect, the functional disruption of the selected gene results in the removal of the entire open reading frame of the selected gene.

In an aspect, “introduced” refers to the introduction by means of modern biotechnology, and not a naturally occurring introduction.

In an aspect, “introduced genetic material” means genetic material that is added to, and remains as a component of, the genome of the recipient.

In an aspect, the term “native” or “endogenous” refers to a substance or process/method that may occur naturally in a host cell.

In an aspect, “genetically modified” denotes a host cell (such as, for example, yeast) comprising a heterologous nucleotide sequence.

In an aspect, “isolated nucleic acid” when applied to DNA refers to a DNA molecule that is isolated from the immediate sequence in the naturally occurring genome of the organism from which it originates. In an aspect, an “isolated nucleic acid” also includes non-genomic nucleic acids, such as cDNA or other non-naturally occurring nucleic acid molecules.

In an aspect, the term “cDNA” is a DNA molecule that can be made by reverse transcription from a mature, spliced mRNA molecule obtained from a cell. cDNA lacks intron sequences that are normally present in the corresponding genomic DNA.

In an aspect, “operably linked” refers to a functional linkage between nucleic acid sequences such that the linked promoter and/or regulatory region functionally controls the expression of the coding sequence.

In an aspect, “epigenome modification” refers to a modification or change in one or more chromosomes that affect gene activity and expression that does not derive from a modification of the genome. An epigenome modification relates to a functionally relevant change to the genome that does not involve a change in the nucleotide sequence. Epigenome modifications may include a modification to a histone, such as acetylation, methylation, phosphorylation, ubiquitination, and/or sumoylation. Epigenome modifications may include a modification to DNA, such as methylation.

In an aspect, “production/quantity produced” generally refers to the amount of a non-catabolic compound produced by the genetically modified host cell provided herein. In an aspect, production is expressed as the yield of the non-catabolic compound produced by the host cell. In an aspect, the amount produced is expressed as the productivity of the host cell in producing the non-catabolic compound.

In an aspect, “yield” refers to the amount of non-catabolic compound produced by a host cell, expressed as the amount of non-catabolic compound produced per amount of carbon source consumed by the host cell, by weight.

In an aspect, “yield” refers to the amount of non-catabolic compounds produced per the amount of total reducing sugars added to the fermentation vessel or flask (i.e., grams of non-catabolic products divided by grams of total reducing sugars added, expressed as a percentage).

The total reducing sugar is a measure of the sugar in grams. A reducing sugar is any sugar that can be used as a reducing agent because it has a free aldehyde group or a free ketone group. All monosaccharides, such as galactose, glucose and fructose are reducing sugars. For example, if 10 grams of a non-catabolic compound is produced by adding 100 grams of glucose (i.e., 100 grams of reducing sugar) to the host cell, the yield per reducing sugar product is 10%.

In an aspect, “productivity” refers to the amount of non-catabolic compound produced by a host cell, expressed as the amount of non-catabolic compound produced per amount of fermentation broth (by weight) in which the host cell is cultured (by volume) over time (per hour).

In an aspect, “fermentation” refers to the cultivation of a host cell that utilizes a carbon source (e.g., sugar) as an energy source to produce a desired product.

For example, fermentation is a process in which yeast metabolizes monosaccharides in wort to ethanol and carbon dioxide. However, these ingredients contribute relatively little to the overall beer flavor.

The aroma and flavor characteristics of beer resulting from a slight flavor volatile component are produced by yeast during fermentation. Another factor is hops. There are two types of hops used for brewing: bitter hops and aroma hops. Bitter hop is used in lager beer to give the beer a further bitter taste. Aroma hops are used in special beers to improve the flavor.

In an aspect, bioflavoring can improve beverage sensory characteristics and develops new beverages. This technology can comprise in the production and conversion of flavor compounds and flavor precursors by biological methods such as the use of special yeast strains.

In an aspect, “medium” refers to a medium that allows the growth of a cellular biomass and the production of metabolites by the host cell. It contains carbon source, and may further contain nitrogen source, phosphorus source, vitamin source, mineral source, etc.

In an aspect, the term “fermentation medium” can be used synonymously with “culture medium”. In general, the term “fermentation medium” can be used to refer to a medium suitable for culturing a host cell for an extended period to produce a desired compound.

The term “medium” refers to a culture medium and/or a fermentation medium. The “medium/vehicle” can be a liquid or a semi-solid. A given medium/media can be both a culture medium and a fermentation medium.

The term “whole cell broth” refers to the entire contents of a vessel (e.g., flask, plate, fermentor, etc.), including cells, an aqueous phase, compounds produced in a hydrocarbon phase and/or emulsion. Thus, whole cell fluids include mixtures of media containing water, carbon sources (e.g., sugars), minerals, vitamins, other dissolved or suspended substances, microorganisms, metabolites, and compounds produced by the host cells, as well as all other components of the material held in a vessel in which non-catabolic compounds are produced by the host cells.

In an aspect, “fermentation composition” is used interchangeably with “whole cell broth”. The fermentation composition may also include a cover if the fermentation composition is added to the fermentation vessel during fermentation.

In an aspect, “fermenting” and “fermentation” means the production of alcohol and carbon dioxide from the breakdown of the carbohydrates by the yeast.

In an aspect, “maturing” and “maturation” means the time following the fermentation when the yeast can continue to affect the flavor and aroma of the beverages through their metabolic activity. Other interchangeable terms for this process include “conditioning” or “secondary fermentation.”

In an aspect, a combination of yeasts can be used in the fermenting and/or maturing steps.

In an aspect, a disclosed method can be provided comprising fermenting at least one carbohydrate in the presence of yeast, wherein the yeast comprises, consists essentially of, or consists of L. thermotolerans strains YB16 and/or BB202, and can further comprise Saccharomyces cerevisiae and/or Saccharomyces pastorianus, to produce a primary fermentation product; and maturing the primary fermentation product (secondary fermentation) to produce a fermented beverage.

In an aspect, a disclosed method of producing a fermented beverage is provided, comprising: fermenting at least one carbohydrate to produce a primary fermentation product; and maturing the primary fermentation product in the presence of yeast, wherein the yeast comprises, consists essentially of, or consists of L. thermotolerans strains YB16 and/or BB202, and further comprises S. cerevisiae and/or S. pastorianus, to produce a fermented beverage.

In an aspect, the at least one carbohydrate is not grapes and/or the fermented beverage is not wine.

In an aspect, disclosed herein is a method for producing mead, comprising fermenting honey in the presence of L. thermotolerans under suitable conditions for a period of time sufficient to produce a fermented beverage.

In an aspect, disclosed herein is a method for producing mead, comprising: fermenting honey in the presence of yeast strain YB16 and/or yeast strain BB202 under suitable conditions for a period of time sufficient to produce mead.

In an aspect, a fermented beverage can be beer, including but not limited to, ale or lager.

In an aspect, a beer can be an American Wheat beer (e.g., light-bodied, crisp refreshing ale; the proportion of wheat is often greater than 50% and the hop aroma and flavor can vary). In an aspect, a beer can be a Barleywine beer (e.g., the richest and strongest of British ales. Typically enjoyed in a snifter for its warming qualities; high alcohol content). In an aspect, a beer can be a Witbier beer (e.g., Belgian unfiltered wheat beer; spiced often with orange and coriander; tangy and sharp from the wheat and high carbonation; slightly hazy due to lack of filtration). In an aspect, a beer can be a Dry Stout beer (e.g., the distinguishing feature of this dark Irish beer is the use of roasted barley to produce a roasted, coffee-like dryness). In an aspect, a beer can be a Lambic beer (e.g., a sour wheat Belgian beer; often made with whole fruit; understated malt and hop characters allow the fruit to remain prominent). In an aspect, a beer can be a Bohemian Pilsner beer (e.g., crisp, light lager with distinct hop flavors; creamy, dense head and well-carbonated; debuted in 1842 in Czechoslovakia by Josef Groll). In an aspect, a beer can be a Porter beer (e.g., a well-hopped beer made from brown malt. Similar to stout with coffee-like dryness and deep malt; production virtually ceased in the 50s until its revival in the 70s). In an aspect, a beer can be Bock beer (e.g., a bottom fermenting lager that takes extra months of lagering to smooth out; by German law, bocks must be of at least 1.064 gravity). In an aspect, a beer can be a Weizen beer (e.g., unfiltered German wheat ale; at least 50% wheat malt, sometimes with more pronounced citrus and spice flavors). In an aspect, a beer can be a Dubbel beer (e.g., strong, malty beer with a notable fruitiness, fairly heavy body, and low bitterness; brewed first in Belgium in 1856 by Trappist monks as a stronger version of a brown beer). In an aspect, a beer can be an Oatmeal Stout beer (e.g., stout that is firm, smooth and silky form the addition of oats to the mash; the smoothness comes from the high content of proteins, and the oats give a sweetness that is unlike any other stout). In an aspect, a beer can be a Christmas Ale beer (e.g., a stronger, darker spiced beer that often has a rich body and warming finish suggesting a good accompaniment for the cold winter season). In an aspect, a beer can be a Marzen/Oktoberfest beer (e.g., ‘March’ beer originally brewed in the spring and stored in ice caves in the summer for autumn celebrations; strong with a malty sweetness and low hop flavor). In an aspect, a beer can be an India Pale Ale (e.g., pale ale with more pronounced hop character and higher alcohol content; Originally brewed in England for the long trip to India). In an aspect, a beer can be Bitter beer (e.g., strong, British and traditionally cask-conditioned; regional preferences create some meltier, stronger ales, and some that are more aggressively hopped and carbonated).

In an aspect, the ale or lager can be sour-style beer.

In an aspect, the lager can be sour-style beer (e.g., when the fermentation occurs at a low temperature).

In an aspect, the fermented beverage can be alcoholic cider or mead (e.g., fermenting honey).

In an aspect, the fermented beverage can be wine or it can be mead.

In an aspect, the fermented beverage is not wine. Methods for brewing these and other different types of fermented beverages are known in the art.

The fermented beverage brewing process comprises, in general, malting, mashing, lautering, boiling, fermenting, maturing (conditioning), filtering and packaging.

As understood by those of skill in art, some of these steps can be skipped and others added, as well as steps can be modified to achieve the particular desired fermented beverage.

Fermentation to produce a beverage can comprise (1) a primary fermentation in which the yeast ferment the at least one carbohydrate selected by the brewer to produce a primary fermentation product, and (2) a maturing step (i.e., secondary fermentation, conditioning or aging), which allows the fermentation process to continue acting on the primary fermentation product, typically in the presence of a reduced and/or modified yeast population. The maturing process can be carried out under the same or different conditions (e.g., temperature) than that used for the fermenting step.

In an aspect, a change in the temperature as compared to the primary fermentation step can be an increase or decrease in temperature. In an aspect, the production of a fermented beverage can comprise a primary fermenting step but not a maturing step. Instead, the process can be completed, and the fermented beverage produced after the fermenting step.

In an aspect, the step of fermenting at least one carbohydrate can be carried out for about 1 day to about 30 days (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 days, or more than about 30 days).

In an aspect, the step of fermenting at least one carbohydrate can be carried out for about 2 days to about 30 days (e.g., about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 days, or more than about 30 days).

In an aspect, the step of fermenting at least one carbohydrate can be carried out for about 2 days to about 21 days (e.g., about 2, about 3, about 4, about 5, about 6, about 7, about 8, about 9, about 10, about 11, about 12, about 13, about 14, about 15, about 16, about 17, about 18, about 19, about 20, about 21 days, or any range or value therein).

In an aspect, the step of fermenting carbohydrates can be carried out at a temperature of about 4° C. to about 36° C. (e.g., about 4° C., about 5° C., about 6° C., about 7° C., about 8° C., about 9° C., about 10° C., about 11° C., 12° C., 13° C., about 14° C., 15° C., 16° C., 17° C., about 18° C., about 19° C., 20° C., 21° C., 22° C., 23° C., 24° C., 25° C., 26° C., 27° C., about 28° C., about 29° C., about 30° C., about 31° C., about 32° C., about 33° C., about 34° C., about 35° C., about 36° C., or any range or value therein, or more than about 36° C.).

In an aspect, the step of fermenting at least one carbohydrate can be carried out at temperature of about 8° C. to about 14° C., or about 18° C. to about 24° C., or about 24° C. to about 36° C., or. Any combination of temperature and time for fermenting the at least one carbohydrate can be chosen by the brewer.

Similar to the choice of time and temperature for fermenting at least one carbohydrate, the selection of temperature and time for maturing the primary fermentation product can depend on the type of beer and the characteristics of the beer desired by the brewer.

For example, when producing lagers, generally the temperature can be reduced in the maturing step as compared to the fermenting step. When producing ales, for example, the temperature chosen for maturing can be about the same or can be lower than that for the fermenting step and for sour-style beers, the temperature for maturing can be highly variable.

In an aspect, maturing can be carried out at a temperature of about 1° C. to about 24° C. (e.g., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or any range or value therein, or more than about 36° C.).

In an aspect, the period for maturing can be from about 10 minutes to about 16 weeks (e.g., about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 35 min, about 40 min, about 45 min, about 50 min, about 55 min, about 1 hr, about 1.5 hr, about 2 hr, about 2.5 hr, about 3 hr, about 3.5 hr, about 4 hr, about 4.5 hr, about 5 hr, about 5.5 hr, about 6 hr, about 6.5 hr, about 7 hr, about 7.5 hr, about 8 h, about 9 hr, about 10 hr, about 11 hr, about 12 hr, about 15 hr, about 18 hr, about 22 hr, about 1 day, about 2 days, about 3 days, about 4 days, 5 days, about 6 days, about 1 week, about 2 weeks, about 3 weeks, about 4 weeks, about 5 weeks, about 6 weeks, about 7 weeks, 8 weeks, about 9 weeks, about 10 weeks, about 11 weeks, about 12 weeks, about 13 weeks, about 14 weeks, about 15 weeks, about 16 weeks and the like, or any range or value therein, or more than 16 weeks).

In an aspect, the period for maturing can be from about 2 days to 21 days (3 weeks) (e.g., about 2, about 3, about 4, about 5, about 6, about 7, about 8, about 9, about 10, about 11, about 12, about 13, about 14, about 15, about 16, about 17, about 18, about 19, about 20, about 21 days and the like, or any range or value therein), or about 10 days to about 12 weeks (e.g., about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or any range or value therein, or more than 12 weeks).

In an aspect, the period for maturing can be up to about 3 months.

In an aspect, the period for maturing a sour-style beer and/or an ale can be, for example, for about 2 days to about 30 days (e.g., about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 days, or any range or value therein, or more than 30 days).

In an aspect, the period for maturing can be about 4 weeks to about 12 weeks, for example, for maturing a lager beer. In an aspect, a brewer can select any combination of temperature and time for maturing the primary fermentation product with the choice being determined by the desired characteristics for the fermented beverage being brewed (e.g., aroma, flavor, foam stability, acidity, alcohol content, mouth feel, or any combination thereof).

Flocculation refers to the tendency of yeast to form clumps called “flocs” that drop as fermentation finishes. Yeast flocculation can be classified as high, medium, or low. Highly flocculating yeasts sink to the bottom of the fermenter more quickly and produce a clearer beer.

In an aspect, beer yeasts can comprise Aurora (Kveik Yeast), Classic American (Yeast), Classic English (Yeast), Funk Weapon #1 (Yeast), Funk Weapon #2 (Yeast), Funk Weapon #3 (Yeast), NEEPAH Blend (Yeast), OSLO (Kveik Yeast), S. arlingtonesis (Yeast), Saison Parfait (Yeast), Sour Solera Blend (Blend), Sour Weapon L (Bacteria), Sour Weapon P (Bacteria), The Mad Fermentationist Saison Blend (Blend), CellarScience Berlin (Yeast), CellarScience Cali (Yeast), CellarScience English (Yeast), CellarScience German (Yeast), CellarScience Nectar (Yeast), Berliner blend (Hybrid), BugFarm (Hybrid), Farmhouse Brett (Yeast), Flemish Ale (Hybrid), Kellerbier (Yeast), Kolschbier (Yeast), Nordic Farmhouse (Kveik Yeast), North East Ale (Yeast), Old Newark Ale (Yeast), Saison Brasserie Blend (Yeast Blend), Scottish Heavy (Yeast), American Ale (Yeast), Anchorman Ale (Yeast), Ardennes Belgian Ale (Yeast), Arset Kveik Blend (Kveik Yeast), Belgian Sour Blend (Yeast), Berliner Brett I (Yeast), Biergarten Lager (Yeast), Brett B (Yeast), Brett D (Yeast), Brett Q (Yeast), Brussels Brett (Yeast), Cali Ale (Yeast), Cerberus (Yeast), Classic Witbier (Yeast), Copenhagen Lager (Yeast), Czech Lager (Yeast), Dry Belgian Ale (Yeast), Ebbegarden Kveik Blend (Kveik Yeast), English Ale I (Yeast), English Ale II (Yeast), English Ale III (Yeast), Foggy London Ale (Yeast), Fruit Bomb Saison (Yeast), Fruity Witbier (Yeast), Hornindal Kveik Blend (Kveik Yeast), Irish Ale (Yeast), Isar Lager (Yeast), Kolsch Ale (Yeast), Krispy (Kveik Yeast), Lactobacillus Blend 2.0 (Hybrid), Lactobacillus brevis (Hybrid), Mexican Lager (Yeast), Munich Lager (Yeast), New World Saison (Yeast), Old World Saison Blend (Yeast), Ontario Farmhouse Ale Blend (Yeast), Saison Maison (Yeast), Skare Kveik (Kveik Yeast), Spooky Saison (Yeast), St-Remy Abbey Ale (Yeast), St. Lucifer Belgian Ale (Yeast), Stirling Ale (Yeast), Uberweizen (Yeast), Vermont Ale (Yeast), Voss Kveik (Kveik Yeast), Weizen I (Yeast), Wild Thing (Yeast), SafAle BE-134 (Yeast), SafAle BE-256 (Yeast), SafAle F-2 (Yeast), SafAle K-97 (Yeast), SafAle S-04 (Yeast), SafAle S-33 (Yeast), SafAle T-58 (Yeast), SafAle US-05 (Yeast), SafAle WB-06 (Yeast), SafBrew DA-16 (Yeast), SafBrew HA-18 (Yeast), SafBrew LA-01 (Yeast), SafLager S-189 (Yeast), SafLager S-23 (Yeast), SafLager W-34/70 (Yeast), SafSour LP 652 (Bacteria), A Touch of Spice Belgian Ale (Yeast), Altstadt Ale (Yeast), American Lager (Yeast), Bavarian Hefe (Yeast), Belgian Abbey Ale (Yeast), Belgian Mix (Yeast), Belgian Tripel (Yeast), Belgian Wit (Yeast), Berliner Blend (Blend), British Ale #1 (Yeast), British Ale #2 (Yeast), British Haze (Yeast), Brussels Bruxellensis (Yeast), Brux Blend (Blend), Czech Pilsner (Yeast), Farmhouse Sour (Blend), German Ale Yeast (Yeast), German Lager (Yeast), Gigayeast Lacto (Bacteria), Golden Gate Lager (Yeast), Golden Pear Belgian (Yeast), Hornindal Kveik #5 (Kveik Yeast), Irish Stout (Yeast), Kolsch Bier (Yeast), Kveik #1 (Kveik Yeast), Norcal Ale #1 (Yeast), Norcal Ale #5 (Yeast), Portland Hefe (Yeast), Quebec Abbey Ale (Yeast), Saison #1 (Yeast), Saison #2 (Yeast), Saison Blend (Yeast), Saison Sour (Blend), Scotch Ale #1 (Yeast), Sour Cherry Funk Blend), Sour Plum Belgian (Blend), Sweet Flemish Brett (Yeast), Tart Cherry Brett (Yeast), Vermont Ipa (Yeast), Barbarian (Yeast), Bartleby Kveik (Yeast), Cablecar (Yeast), Citrus (Yeast), Darkness (Yeast), Dieter (Yeast), Dry Hop (Yeast), Flagship (Yeast), Global (Yeast), Gnome (Yeast), Harvest (Yeast), House (Yeast), Independence (Yeast), Joystick (Yeast), Juice (Yeast), Kaiser (Yeast), Kveiking (Kveik Yeast), Loki (Kveik Yeast), Napoleon (Yeast), POG (Kveik Yeast), Pub (Yeast), Rustic (Yeast), Sour Batch Kidz (Hybrid), Stefon (Yeast), Suburban Brett (Yeast), Tartan (Yeast), Triple Double (Yeast), Urkel (Yeast), Whiteout (Yeast), LalBrew Abbaye (Yeast), LalBrew Belle Saison (Yeast), LalBrew BRY-97 (Yeast), LalBrew CBC-1 (Yeast), LalBrew Diamond Lager (Yeast), LalBrew Koln (Yeast), LalBrew London (Yeast), LalBrew Munich Classic (Yeast), LalBrew New England (Yeast), LalBrew Nottingham (Yeast), LalBrew Verdant IPA (Yeast), LalBrew Voss (Kveik Yeast), LalBrew Windsor (Yeast), LalBrew Wit (Yeast), Prise de Mousse Wine Yeast (Yeast), Sourvisiae (Yeast), WildBrew Philly Sour (Yeast), Bavarian Wheat (Yeast), Belgian Ale (Yeast), Belgian Wit (Yeast), Bohemian Lager (Yeast), California Lager (Yeast), Empire Ale (Yeast), French Saison Ale (Yeast), Liberty Bell Ale (Yeast), New World Strong Ale (Yeast), US West Coast (Yeast), Workhorse (Yeast), Muntons Premium Gold (Yeast), Muntons Standard Ale (Yeast), Abbey Ale C (Yeast), All the Bretts (Yeast), ALT (Yeast), American Lager (Yeast), American Pilsner (Yeast), American Wheat (Yeast), Bavarian Wheat I (Yeast), Bavarian Wheat II (Yeast), Bayern Lager (Yeast), Belgian Ale A (Yeast), Belgian Ale D (Yeast), Belgian Ale DK (Yeast), Belgian Ale O (Yeast), Belgian Ale R (Yeast), Belgian Ale W (Yeast), Belgian Golden Strong (Yeast), Belgian Saison I (Yeast), Belgian Saison Ii (Yeast), Belgian Wheat (Yeast), Biere De Garde (Yeast), Brett Blend #1 Where Da Funk? (Yeast), Brett Blend #2 Bit O'Funk (Yeast), Brett Blend #3 Bring On Da Funk (Yeast), Brettanomyces Bruxellensis (Yeast), Brettanomyces Claussenii (Yeast), Brettanomyces Lambicus (Yeast), British Ale I (Yeast), British Ale II (Yeast), British Ale III (Yeast), British Ale IV (Yeast), British Ale V (Yeast), British Ale VI (Yeast), British Ale VII (Yeast), British Ale VIII (Yeast), C2C American Farmhouse (Blend), CL-50 Ale (Yeast), Danish Lager (Yeast), Dipa Ale (Yeast), East Coast Ale (Yeast), Espe Kveik (Kveik Yeast), French Saison (Yeast), German Bock (Yeast), German Lager I (Yeast), German Lager II (Yeast), Grand Cru (Yeast), Gulo Ale (Hybrid), Hefeweizen Ale I (Yeast), Hefeweizen Ale II (Yeast), Hornindal Kveik (Kveik Yeast), Hothead Ale (Kveik Yeast), Irish Ale (Yeast), Jovaru Lithuanian Farmhouse (Yeast), Kolsch I (Yeast), Kolsch Ii (Yeast), Lactobacillus Blend (Blend), Lager I (Yeast), London Ale (Yeast), Lutra Kveik (Kveik Yeast), Mexican Lager (Yeast), Northwest Farmhouse Brett (Yeast), Oktoberfest (Yeast), Pacific NW Ale (Yeast), Pediococcus (Blend), Pilsner I (Yeast), Pilsner II (Yeast), Saisonstein's Monster (Hybrid), Scottish Ale (Yeast), Tropical IPA (Yeast), Voss Kveik (Kveik Yeast), West Coast Ale I (Yeast), West Coast Ale II (Yeast), West Coast Ale III (Yeast), West Coast Ale IV (Yeast), West Coast Lager (Yeast), WIT (Yeast), Chico Ale (Yeast), Chiswick Ale (Yeast), Hoptopper Ale (Yeast), Manchester Ale (Yeast), Oktoberfest Lager (Yeast), Pacman Ale (Yeast), Amalgamation—Brett Super Blend (Yeast), Amalgamation II—Brett Super Blend (Yeast), Beersel Brettanomyces Blend (Yeast), Brettanomyces Bruxellenis TYB261 (Yeast), Brettanomyces Bruxellenis—STRAIN TYB184, Brettanomyces Bruxellensis—Strain TYB207, Brettanomyces Bruxellensis—Strain TYB307, Brettanomyces bruxellensis—Strain TYB415, Brussels Brettanomyces Blend (Yeast), Dark Belgian Cask (Blend), Dry Belgian Ale (Yeast), Farmhouse Sour Ale (Blend), Flanders Specialty Ale (Yeast), Framgarden Kveik (Kveik Yeast), Franconian Dark Lager (Yeast), Funktown Pale Ale (Blend), Hazy Daze (Yeast), Hazy Daze II (Yeast), Hessian Pils (Yeast), Hornindal Kveik (Kveik Yeast), Lactobacillus Blend (Bacteria), Lactobacillus brevis—Strain TYB282 (Bacteria), Lida Kveik (Kveik Yeast), Lochristi Brettanomyces (Blend), M61ange—Sour Blend Hybrid), Metschnikowia reukaufii, Midtbust Kveik (Kveik Yeast), Midwestern Ale (Yeast), Northeastern Abbey (Yeast), Pakruojis Lithuanian Farmhouse (Kveik Yeast), Saison Blend (Blend), Saison Blend II (Yeast), Saison/Brettanomyces Blend (Yeast), Saison/Brettanomyces Blend II (Blend), Sigmund's Voss Kveik (Kveik Yeast), Sigmund's Voss Kveik (Kveik Yeast), Simonaitis Lithuanian Farmhouse (Kveik Yeast), Transatlantic Berliner Blend (Hybrid), Vermont Ale (Yeast), Wallonian Farmhouse (Yeast), Wallonian Farmhouse II (Yeast), Wallonian III Farmhouse (Yeast), Abbey Ale Yeast (Yeast), Abbey IV Ale Yeast (Yeast), American Ale Yeast Blend (Blend), American Farmhouse Blend (Yeast), American Hefeweizen Ale Yeast (Yeast), American Lager Yeast (Yeast), Bastogne Belgian Ale Yeast (Yeast), Bavarian Weizen Ale Yeast (Yeast), Bedford British Ale (Yeast), Belgian Ale Yeast (Yeast), Belgian Golden Ale Yeast (Yeast), Belgian Saison I Ale Yeast (Yeast), Belgian Saison II Ale Yeast (Yeast), Belgian Strong Ale Yeast (Yeast), Belgian Wit Ale Yeast (Yeast), Belgian Wit II Ale Yeast (Yeast), Belgian-Style Ale Yeast Blend (Yeast), Belgian-Style Saison Ale Yeast Blend (Blend), Brettanomyces Claussenii (Yeast), Brettanomyces Lambicus (Yeast), British Ale Yeast (Yeast), Burlington Ale Yeast (Yeast), Burton Ale Yeast (Yeast), California Ale Yeast (Yeast), California V Ale Yeast (Yeast), Charlie's Fist Bump Yeast (Yeast), Coastal Haze Ale Yeast Blend (Blend), Copenhagen Lager Yeast (Yeast), Cream Ale Yeast Blend (Yeast), Czech Budejovice Lager Yeast (Yeast), Dry English Ale Yeast (Yeast), Dusseldorf Alt Ale Yeast (Yeast), East Coast Ale Yeast (Yeast), East Midlands Ale Yeast (Saccharomyces), Edinburgh Scottish Ale Yeast (Yeast), English Ale Yeast (Yeast), French Saison Ale Yeast (Yeast), German Bock Lager Yeast (Yeast), German Lager Yeast (Yeast), German/Kolsch Ale Yeast (Yeast), Hefeweizen Ale Yeast (Yeast), Hefeweizen IV Ale Yeast (Yeast), High Pressure Lager Yeast (Yeast), Hornindal Kveik Kveik (Yeast), Irish Ale Yeast (Yeast), Lactobacillus Brevis (Bacteria), Lactobacillus delbrueckii (Bacteria), London Ale Yeast (Yeast), London Fog Ale Yeast (Yeast), Mexican Lager Yeast (Yeast), Monastery Ale Yeast (Yeast), Oktoberfest/Marzen Lager Yeast (Yeast), Old Bavarian Lager Yeast (Yeast), Opshaug Kveik Ale Yeast (Kveik Yeast), Pacific Ale Yeast (Yeast), Pilsner Lager Yeast (Yeast), Saccharomyces “Bruxellensis” Trois (Yeast), San Diego Super Ale Yeast (Yeast), San Francisco Lager Yeast (Yeast), Southern German Lager Yeast (Yeast), Super High Gravity Ale Yeast (Yeast), American Ale (Yeast), American Ale II (Yeast), American Lager (Yeast), American Wheat (Yeast), Bavarian Lager (Yeast), Bavarian Wheat (Yeast), Bavarian Wheat Blend (Yeast Blend), Belgian Abbey Style Ale (Yeast), Belgian Abbey Style Ale II (Yeast), Belgian Ardennes (Yeast), Belgian Dark Ale (Yeast), Belgian High Gravity (Yeast), Belgian Lambic Blend (Hybrid), Belgian Saison (Yeast), Belgian Schelde Ale (Yeast), Belgian Stout (Yeast), Belgian Strong Ale (Yeast), Belgian Style Blend (Yeast Blend), Belgian Wheat (Yeast), Belgian Witbier (Yeast), Berliner Weisse Blend (Hybrid), Biere de Garde (Yeast), Bohemian Lager (Yeast), Brettanomyces Bruxellensis (Yeast), Brettanomyces Lambicus (Yeast), British Ale (Yeast), British Ale II (Yeast), British Cask Ale (Yeast), Budvar Lager (Yeast), Burton IPA Blend (Yeast Blend), California Lager (Yeast), Canadian/Belgian Ale (Yeast), Czech Pils (Yeast), Danish Lager (Yeast), Denny's Favorite 50 Ale (Yeast), English Special Bitter (Yeast), European Lager (Yeast), Farmhouse Ale (Yeast), Flanders Golden Ale (Yeast), Forbidden Fruit (Yeast), French Saison (Yeast), Gambrinus Style Lager (Yeast), German Ale (Yeast), German Wheat (Yeast), Hella Bock Lager (Yeast), Irish Ale (Yeast), Kolsch (Yeast), Kolsch II (Yeast), Lactobacillus buchneri (Bacteria), London Ale (Yeast), London Ale III (Yeast), London ESB Ale (Yeast), Munich Lager (Yeast), Munich Lager II (Yeast), North American Lager (Yeast), Northwest Ale (Yeast), Octoberfest Lager Blend (Yeast Blend), Old Ale Blend (Yeast Blend), Oud Bruin Ale Blend (Hybrid), Pacman (Yeast), Pilsen Lager (Yeast), Pilsner Urquell H-Strain (Yeast), Ringwood Ale (Yeast), Rocky Mountain Lager (Yeast), Roeselare Ale Blend (Hybrid), Saison-Brett Blend (Yeast Blend), Scottish Ale (Yeast), Staro Prague Lager (Yeast), Thames Valley Ale (Yeast), Thames Valley Ale II (Yeast), Weihenstephan Weizen (Yeast), West Coast IPA (Yeast), West Yorkshire Ale (Yeast), Whitbread Ale (Yeast), Wyeast Bohemian Ale Blend (Yeast Blend), or any combination thereof.

In an aspect, “yeast species” refers to a recognized biological group as determined by the International Code of Botanical Nomenclature (see, Seifert and Rossman (2010) IMA Fungus. 1(2):109-116).

In an aspect, “yeast strain” refers to a taxonomic grouping which is one lower than species. These are unique, or putatively unique, clones that may vary from one another in genotype and phenotype, but are still more closely related to one another (as a species group), than to the strains of another species.

Beer styles have diverged over the past two hundred years into two main categories, ale and lager, their differences due primarily to the species of yeast used for fermentation, S. cerevisiae or S. pastorianus. Typically, ale is fermented at room temperatures (18° C.-24° C.) by S. cerevisae with short maturation times (typically 10-30 days) and the beer characterized by some residual sweetness and moderate to high bitterness. In contrast, lagers are fermented at cellar temperatures (8° C.-14° C.) by S. pastorianus with long maturation times (4-12 weeks), and the beer characterized by minimal residual sweetness and low to moderate bitterness.

Belgian sour-style beer, referred to as lambic or gueuze, is produced by mixed culture fermentations of wort that, in addition to barley malt, usually contains some wheat and/or fruit and aged (oxidized) hops. The culture responsible for the fermentation is often based on the local cultures found within the specific brewery environment and can be composed of species from the genera of Saccharomyces, Brettanomyces, Dekkera, Kloeckera, Pediococcus and Lactobacillus (Guinard 1990). The fermentations for sour-style beers are for extended times, typically several months to 2 years for traditional lambics. This type of beer is characterized by high levels of organic acids (mainly lactic and acetic with lesser amounts of propionic, isobutyric and butyric) with a pH ranging between 3.3 to 3.9. Ethyl acetate is also found at elevated levels in these beers, adding to the vinegar odor. The unique composition of the wort, combined with the variety and types of microbes used as well as the long fermentation times, results in very different flavor profiles from that of standard ales and lagers.

In an aspect, “biosynthetic pathway” refers to a pathway having a set of anabolic or catabolic biochemical reactions for converting one chemical into another, thereby producing a molecule. Gene products belong to the same “biosynthetic pathway” if they act in parallel or in series on the same substrate, producing the same product, or on or producing a metabolic intermediate (e.g., metabolite) between the same substrate and the metabolite end product.

In an aspect, “biosynthetic pathway” refers to a pathway having a set of anabolic or catabolic biochemical reactions for converting one chemical into another, thereby producing a molecule. Gene products belong to the same “biosynthetic pathway” if they act in parallel or in series on the same substrate, producing the same product, or on or producing a metabolic intermediate (e.g., metabolite) between the same substrate and the metabolite end product.

In an aspect, “promoter” refers to a nucleic acid of synthetic or natural origin which is capable of conferring, activating or enhancing the expression of a DNA coding sequence. A promoter may comprise one or more specific transcriptional regulatory sequences to further enhance expression and/or alter spatial and/or temporal expression of the coding sequence. The promoter can be located 5′ (upstream) of the coding sequence under its control. The distance between the promoter and the coding sequence to be expressed can be about the same as the distance between the promoter and the native nucleic acid sequence it controls. Variations in the distance can be accommodated without loss of promoter function. In an aspect, the regulatory promoter generally allows transcription of a nucleic acid sequence encoding a transcriptional regulator (e.g., an activator such as Gal4p, or an inhibitor such as Gal80 p) in a permissive environment (e.g., in the presence of maltose), but stops transcription of the nucleic acid sequence encoding the transcriptional regulator in a non-permissive environment (e.g., in the absence of maltose).

In an aspect, “maltose-responsive promoter” or “pMAL” promoter refers to a promoter sequence that is bound by and regulated by a transcriptional activator that is regulated by maltose. For example, a maltose-inducible promoter is regulated by an MAL operon activator (e.g., MAL transcription activator) or a functional homolog thereof. In an aspect, “MAL operon activator” or “MAL transcription activator” refers to a DNA-binding, maltose-dependent transcription activator of a maltose operon or a maltose-responsive promoter. In an aspect, “galactose-inducible promoter” or “pGAL” promoter refers to a promoter sequence that is bound by and regulated by a transcriptional activator regulated by galactose. In an aspect, “pGMAL” promoter refers to a synthetic promoter having a pGAL promoter sequence whose GAL transcriptional activator (e.g., GAL4 p) binding site is replaced with one or more binding sites of MAL transcriptional activator. Thus, the pGMAL promoter is activated by MAL transcriptional activator, not GAL transcriptional activator. In an aspect, “synthetic promoter” refers to a nucleotide sequence that has promoter activity and is not known in nature. In an aspect, the synthetic promoter is assembled from multiple elements that are heterologous to each other.

In an aspect, “strain stability” generally refers to the stability of a host cell genetically modified as described herein to produce a heterologous compound over a prolonged period of fermentation. In particular, stability refers to the ability of a microorganism to maintain the favorable production characteristics of a non-catabolic fermentation product (i.e., high yield (grams of compound per gram of substrate) and/or productivity (grams per liter of fermentation broth per hour)) over an extended culture period, e.g., about 3 to 20 days. Genetic stability, which refers to the tendency of the producing microbiota to hardly change the expected allele frequencies of the genes associated with the production of the product over time, plays a major role in the continued production of the product.

The concentration units of maltose or other components in the culture medium or culture solution are weight/volume percentages. Defined as solute concentration (w/v %)=(solute weight (g)/solution volume (mL))×100.

In an aspect, “transcriptional regulator” refers to a protein that controls gene expression. In an aspect, “transcriptional activator” refers to a transcriptional regulator that activates or positively regulates gene expression. In an aspect, “transcription repressing factor” refers to a transcription regulatory factor that represses or negatively regulates gene expression.

In an aspect, “gene that affects cell growth” or “nucleic acid encoding a protein that affects cell growth” refers to a nucleic acid that encodes a protein that affects cell growth (e.g., growth rate or cell biomass) of a cell.

In an aspect, “essential gene” refers to a gene absolutely required to sustain life under optimal conditions where all nutrients are available. In an aspect, “conditionally essential gene” refers to a gene that is only essential under certain conditions or growth conditions.

In an aspect, “regulator” refers to a genome or group of nucleic acids that is regulated by the same regulatory protein (e.g., a transcriptional regulator). The genes of the regulon have regulatory binding sites or have promoters that are regulated by common transcriptional regulators.

The genome or nucleic acid set comprising the regulon can be located continuously or discontinuously in the genome of the host cell.

In an aspect, “inducible promoter” refers to a promoter that is activated by an inducer to induce transcription of a gene it controls.

In an aspect, “constitutive promoter” refers to a promoter that does not require the presence of an inducer to induce transcription of the gene it controls.

In an aspect, “expression” refers to the production of mRNA by transcription of the gene of interest and/or by transcription of the gene to produce a protein, followed by translation of the mRNA.

In an aspect, “catabolism” refers to a process/method of molecular breakdown or degradation of a large molecule into smaller molecules.

In an aspect, “non-catabolic” refers to processes/methods of constructing molecules from smaller units, and these reactions typically require energy. The term “non-catabolic compound” refers to a compound produced by a non-catabolic process.

In an aspect, a target gene can be inaccessible to one or more transcriptional control sequences.

In an aspect, a transcriptional control sequence can regulate, modulate, or influence expression of one or more target genes. Target genes are disclosed herein and included, but are not limited to, ADH1-ADH7, (alcohol dehydrogenase); ALD (alanine dehydrogenase); ARO10 (phenylpyruvate decarboxylase); BAT1 (branched-chain-amino-acid); transaminase); BAT2 (branched-chain-amino-acid transaminase); CHA1 (1-serine/l-threonine ammonia-lyase); EHT1 (medium-chain fatty acid ethyl ester synthase/esterase), FAA4 (fatty acid activation 4); HOR7; ILV3 (dihydroxy-acid dehydratase); ILV5 (ketol-acid reductoisomerase); ILV6 (acetolactate synthase regulatory subunit); IMA1 (isomaltase); IRA1 (GTPase-activating protein); IRC7; LEU1 (3-isopropylmalate dehydratase); LEU2 (3-isopropylmalate dehydrogenase); LEU4 (2-isopropylmalate synthase); LEU9 (2-isopropylmalate synthase); QCR7 (ubiquinol-cytochrome C oxidoreductase); OLE1 (oleic acid requiring); PDC (pyruvate decarboxylase); PUT1 (proline utilization); SFA1 (bifunctional alcohol dehydrogenase), SSD1, THI3 (branched-chain-2-oxoacid decarboxylase); TPS2 (Trehalose-6-Phosphate Synthase/phosphatase); or any combination thereof.

In an aspect, a disclosed target gene can have a defined state of expression, e.g., expression in its native state and/or expression in a diseased state. In an aspect, a disclosed target gene can have a moderate to low level of expression. In an aspect, a disclosed target gene can have a moderate to high level of expression.

In an aspect, the targeting moiety targets one or more nucleotides, e.g., such as through CRISPR, TALEN, dCas9, oligonucleotide pairing, recombination, transposon, etc., of a gene targeted for gene expression by, for example, substitution, addition, or deletion.

In an aspect, a disclosed targeting moiety can alter one or more nucleotides, such as through a gene editing system, of a sequence in a gene targeted for gene expression by, for example, substitution, addition or deletion.

In an aspect, the activity or expression of target gene can be modulated. In an aspect, modulated means increased or decreased expression of a target gene during any point before, after, or during translation.

In an aspect, activity or expression of a disclosed target gene can be modulated during translation. In an aspect, inhibition of translation of a disclosed target gene can be modulated expression. In an aspect, the expression level of a disclosed target gene can be modulated if the steady-state level of the expressed protein decreased even though translation was not inhibited.

In an aspect, a change in the half-life of a mRNA can modulate expression. In an aspect, modulated activity or expression of a target gene can be increased or decreased expression during any point before, during, or after translation.

In an aspect, the activity or expression of a disclosed target gene can be increased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.).

In an aspect, the activity or expression of a disclosed target gene can be decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.).

In an aspect, the activity or expression of a disclosed target gene can be increased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.) when compared to a non-modulated level of activity or expression (e.g., having no contact and/or exposure with a modulatory technology).

In an aspect, the activity or expression of a disclosed target gene can be decreased by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 20%, or more than at least about 100% following contact and/or exposure with a modulatory technology (e.g., CRISPR, TALEN, dCas9, oligonucleotide pairing, recombination, transposon, etc.) when compared to a non-modulated level of activity or expression (e.g., having no contact and/or exposure with a modulatory technology).

Each beer contains a number of flavor compounds derived from barley malt, hops, yeast fermentation, and other ingredients. However, yeast fermentation is part of the beer brewing process as flavor compounds are formed during fermentation.

In an aspect, reducing the “bad” flavor and improving the desired flavor can be desirable goals or endpoints. The reduction in bad flavor can be focused on the reduction of diacetyl, an undesirable flavor compound of beer with a “butterscotch” odor. In an aspect, desirable flavor compounds of beer can be esters and higher alcohols, which give the beer a fruity aroma.

In an aspect, a disclosed method can generate a desired type of beer. Beer types are known to the skilled person and include, but are not limited to the following:

In an aspect, “American hops” refers to American brewing hops from the craft beer era, typically having citrusy, resiny, evergreen, or similar characteristics. More modem hops can add a wider range of characteristics, such as stone fruit, berry, tropical fruit, and melon.

In an aspect, “Continental hops” or “Old World hops” refers to traditional European brewing hops, including German and Czech landrace hops, British brewing hops, and those other varieties from continental Europe. Typically described as floral, spicy, herbal, or earthy. Generally less intense than many New World hops.

In an aspect, “Dry-Hopped” refers to a post-boil addition of uncooked hop products that gives the beer a fresh, bright hop aroma. A dry-hopped beer is often more robust, vivid, and focused than the same beer without dry hops. It can shift the balance of the beer to be more hop-focused without adding bitterness. Should not be grassy, vegetal, oxidized, cheesy, or old in character. Bright and fresh, not cooked.

In an aspect, “juicy” refers to a trendy modern term used to describe hops that have a quality like fresh fruit juices, especially tropical fruits. Has other meanings, such as “mouth-watering” or “wet” that don't apply in brewing.

In an aspect, “New World hops” refers to American hops, along with those from Australia and New Zealand, and other non-Old World locations. Can have all the attributes of classic American hops, as well as tropical fruit, stone fruit, white grape, and other interesting aromatics.

In an aspect, “Traditional German” or “Czech hops” (also called noble or landrace hops) refers to long considered having the finest, most refined character for traditional European lagers.

Often having a subtle, lightly floral, spicy, or herbal character. Traditional implies that these are classic types, not modern, aggressive hops.

In an aspect, some relevant malt or mashing terms are defined as follows:

In an aspect, biscuity refers to dry, toasted grain, flour, or dough flavor reminiscent of English digestive biscuits or cookies; in brewing, a flavor commonly associated with Biscuit malt and some traditional English malts.

In an aspect, maillard products refer to a class of compounds produced from complex interactions between sugars and amino acids at high temperatures, resulting in brown colors and rich, malty, sometimes even somewhat meaty compounds. In previous versions of the guidelines, known as melanoidins, which are a subset of Maillard products responsible for red-brown colors (and, according to Kunze, are “aroma-intensive”). In some brewing literature, melanoidin and Maillard product are used interchangeably. The chemistry and flavor characterization of Maillard products are not well understood, so brewers and judges should avoid excessively pedantic discussions around these points.

In an aspect, Munich malt refers to a bready, richly malty quality that enhances the malt backbone of a beer without adding residual sweetness, although some can confuse maltiness with sweetness. Darker Munich malts can add a deeply toasted malt quality similar to toasted bread crusts.

In an aspect, “pilsner or pils malt” refers to continental Pilsner malt is quite distinctive, and has a slightly sweet, lightly grainy character with a soft, slightly toasty, honey-like quality. Higher in DMS precursors than other malts, its use can sometimes result in a low corny DMS flavor.

In an aspect, “Vienna malt” refers to can provide a bready-toasty malt presence, but don't expect the toasted notes to be extreme—they're more like the crust of freshly baked bread than toasted bread.

In an aspect, some relevant yeast or fermentation terms are defined as follows:

In an aspect, “Bubblegum” refers to the flavor profile of Bazooka Bubble Gum original flavor, a pink chewing gum; a sweet mixed fruit flavor dominated by banana and strawberry with fruit punch flavors.

In an aspect, “clean fermentation profile” refers to the quality of having very low to no yeast-derived fermentation byproducts in the finished beer, typically implying that there are no esters, diacetyl, acetaldehyde, or similar components, except if specifically mentioned. A shorthand for saying that the long list of possible fermentation byproducts is not present in significant or appreciable quantities (barely perceived trace quantities at the threshold of perception are typically acceptable, nonetheless).

In an aspect, “Kveik” traditionally refers to a mixed blend of yeast in Norway used to produce farmhouse style ales, often available as single strains today. Not a beer style.

In an aspect, “Pome fruit” refers to apple, pear, and quince. The botanical classification contains other fruit, but these are the common ones we mean.

In an aspect, “Stone fruit” refers to a fleshy fruit with a single pit (or stone), such as cherry, plum, peach, apricot, mango, etc.

In an aspect, some relevant mixed fermentation terms are defined as follows:

In an aspect, “ascetic character” refers to vinegar-like, sharp, not a clean sourness.

In an aspect, “Brett” is a shorthand term for Brettanomyces, an attenuative genus of yeast that often is used to produce fruity (pome fruit, tropical fruit, stone fruit), floral, and often funky complex flavors (leather, sweat, barnyard, horse blanket, funk, etc.) in fermented beverages. Derived from phenols or fatty acids produced during fermentation. Literally means “British fungus” and is associated with qualities produced during barrel aging.

Common species used in brewing include B. bruxellensis and B. anomalous, although they are sometimes known by other names; several strains exist with very different profiles (as with S. cerevisiae). Typically used as secondary fermentation strain, although a few strains exist that can fully attenuate wort enough to be used for primary fermentation.

In an aspect, “clean sourness” is a quality descriptor for sourness to imply that the sourness has no vinegar, complex funk, or excessive overtones; often used to describe a good-quality, sharp lactic sourness.

In an aspect, “Ethyl acetate” is a yeast-derived ester formed from acetic acid and ethanol and produced at various levels depending on yeast strain and stress. Low levels are fruity like pears, pineapples, or berries but high levels are objectionable faults and have the aroma of solvent or nail polish remover. High levels of oxygen and wild yeast can create excessive amounts.

In an aspect, “Indole” is formed by ‘coliform’ bacteria contamination during fermentation.

It is often associated with simultaneous production of DMS. Most often found in beers that have a very long lag time or in spontaneous-fermented beer. Smells of feces, dirty farm, or pig farms.

At lower levels, can be jasmine or floral. Always a fault.

In an aspect, “LAB” is shorthand for Lactic Acid Bacteria, including Lactobacillus, Pediococcus, and others in the family Lactobacillaceae. A broader term for identifying the source of a lactic sourness. In an aspect, “Lacto” is shorthand term for Lactobacillus. In an aspect, “Pedio” is shorthand term for Pediococcus. In an aspect, “Sacch” is shorthand term for Saccharomyces.

In an aspect, “ropiness” describes a mouthfeel where the beer develops an increase in viscosity and pours thick and syrupy. Various bacteria are the usual cause, Pedio being most common, and happens from an increase in production of polysaccharides. A common stage in mixed-culture fermentation; the presence of Brett will reduce this viscosity over time.

In an aspect, “THP” is shorthand for tetrahydropyridine. Usually produced by Lacto or Brett. At low levels, lends grainy, toasted oat cereal-like character (think ‘Cheerios’ cereal in the US). At high levels, can be perceived as mouse cage, mousy, or urine-like (similar to the fault in cider and wine). THP increases with oxygen exposure but active Brett will reduce it over time. Always a fault.

In an aspect, some relevant quality or off-flavor terms are defined as follows:

In an aspect, “adjunct quality” refers to a characteristic of beer aroma, flavor, and mouthfeel that reflects the use of higher percentages of non-malt fermentables; can present as a corny character, a lighter body than an all-malt product, or a generally thinner-tasting beer; does not necessarily imply the use of any specific adjunct.

In an aspect, “balanced” refers to to a style, balanced implies a pleasant, harmonious, agreeable, complementary mix of elements, not an equal amount of each component; does not imply any absolute quantity, more of a measure of appropriate coordination of flavor constituents.

In an aspect, “clean” means lacking off flavors; a positive term.

In an aspect, “crisp” refers to a rapid, abrupt change in the mouthfeel of beer from smoothness to sharpness, leading into a dry finish; usually a positive term.

In an aspect, “DMS” means dimethyl sulfide, which can take on a wide range of perceptual characteristics. Most are inappropriate in any style of beer; however, a light, background cooked corn quality may be noted and is acceptable in beers with high levels of Pilsner malt. When the guidelines state that any levels of DMS are appropriate, it is this light cooked corn flavor, not other cooked vegetable characteristics or other DMS flavors.

In an aspect, “dry” is used in the same way with wine, meaning lacking perceived sweetness; Well-attenuated; does not mean “opposite of wet” in this context.

In an aspect, “elegant” refers to a smooth, tasteful, refined, pleasant character suggestive of high-quality ingredients handled with care; lacking rough edges, sharp flavors, and palate-attacking sensations.

In an aspect, “harsh” when applied to beer, an unpleasant, sharp, intense, or disagreeable texture, flavor, or aftertaste; Some synonyms in this context are rough, coarse, abrasive, not fine, dirtier, less refined, and less pure; a quality term indicating the opposite of smooth, clean, and pleasant; can imply astringency, but also can apply to bitterness, alcohol, and other sensations.

In an aspect, “funky” is a positive or negative term, depending on the context. If expected or desirable, can often take on a barnyard, wet hay, slightly earthy, horse blanket, or farmyard character. If too intense, unexpected, or undesirable, can take the form of silage, fecal, baby diaper, or horse stall qualities.

In an aspect, “rustic” refers to coarse, hearty, robust character reminiscent of older, traditional ingredients; perhaps less refined as a general sensory experience.

In an aspect, some relevant appearance terms are defined as follows:

In an aspect, “Belgian Lace, Lacing” refers to a characteristic and persistent latticework pattern of foam left on the inside of the glass as a beer is consumed; the look is reminiscent of fine lacework from Belgium, where it is considered a desirable indicator of beer quality.

In an aspect, “legs” is a pattern that a beverage leaves on the inside of a glass after a portion has been consumed. The term refers to the droplets that slowly fall in streams from beverage residue on the side of the glass; not an indication of quality, but can indicate a higher alcohol, sugar, or glycerol content.

In an aspect, upregulate is a process by which a cell increases its response to a substance or signal from outside the cell to carry out a specific function.

In an aspect downregulate is a process of reducing or suppressing a response to a stimulus.

Strain optimization is a precision game of modifying one of the thousands or so (e.g., 6000 or more) genes of a gene pool of a micro-organism to achieve a target objective while not impacting other genes of the gene pool. As generally known, the proof is in the pudding and success is measured as an output of a fermentation process. Various embodiments in the present disclosure describe using one or more machine learning algorithms on multi-omics datasets about micro-organisms such as, bacteria, fungi, etc., to predict in-silico what the outcome of the fermentation is likely to be. Accordingly, the cost and time required to predict in-silico is less in comparison to actual fermentation to conduct. Additionally, possible optimization options may also be evaluated quickly.

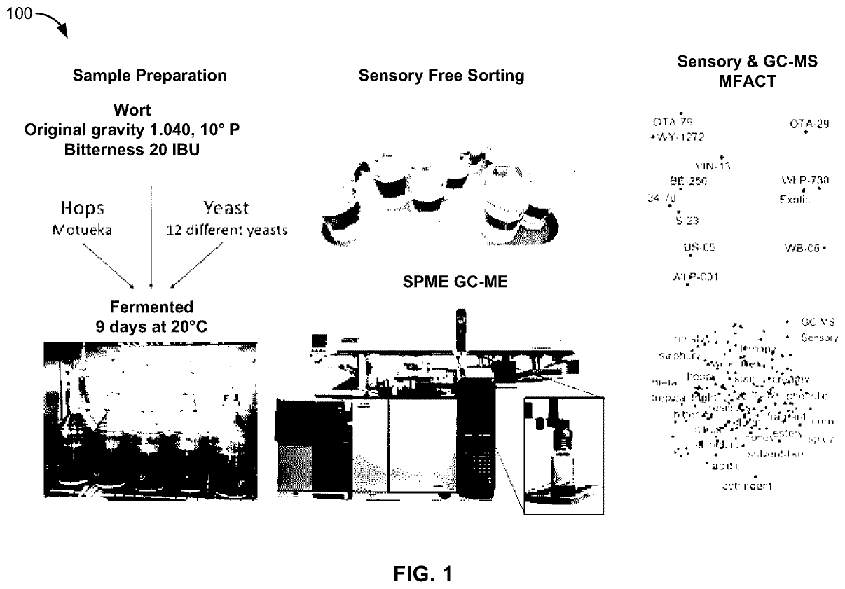

FIG. 1 is an example of a known beer fermentation process 100 for evaluation of different yeast strains (e.g., different variants of the same yeast organism. The beer fermentation process 100 is time and resources consuming due to every investigation into a new strain of yeast needs to be matched with a controlled experiment lasting a few days. Even though only a few documented and commercially available yeast strains are known, the beer fermentation process 100 when considered in the context of gene editing of yeast, the investigation into each new strain of yeast becomes a daunting task. As an example, the quest for a high yield casein-producing gene-editing yeast with a small amount of undesired by-product compounds requires thousands of wet lab experiments.

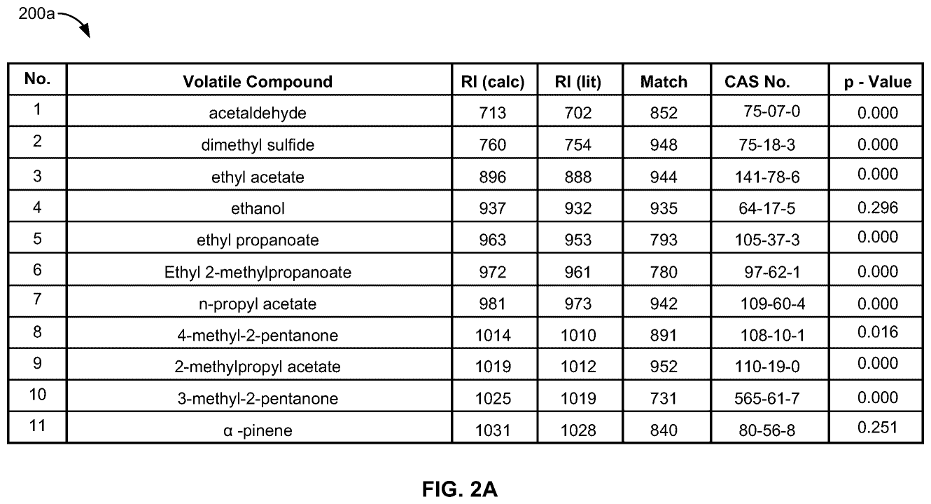

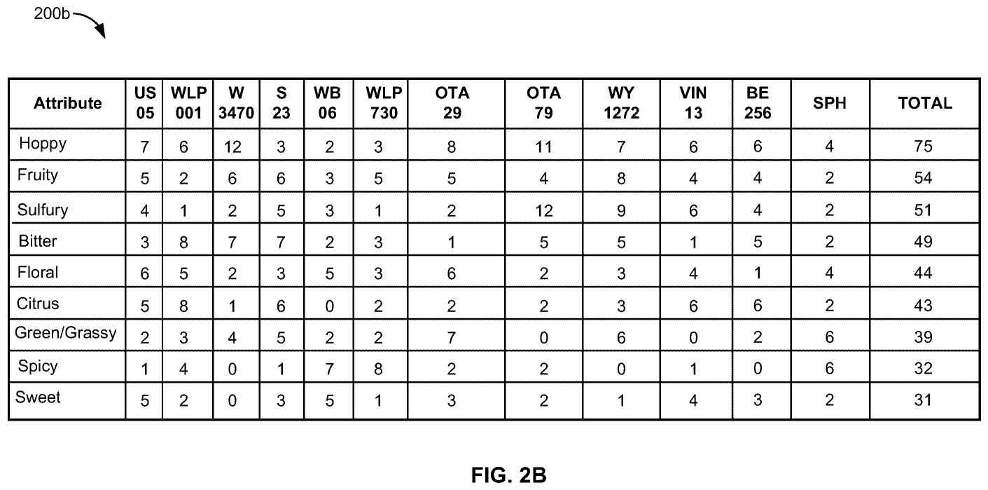

FIG. 2A is a table 200a of volatile organic compounds identified in twelve different beer samples. FIG. 2B is a table 200b showing a qualitative evaluation of twelve different beer samples. Tables 200a and 200b are based upon experiments performed using wet lab experiments on twelve different yeast strains (and therefore two different beer samples). As shown in table 200a, volatile compounds found in the beer samples after the fermentation process include, but not limited to, acetaldehyde, dimethyl sulfide, ethyl acetate, ethanol, ethyl propanoate, ethyl 2-methylpropanoate, n-propyl acetate, 4-methyl-2-pentanone, 2-methylpropyl acetate, 3-methyl-2-pentanone, and/or α-pinene. The qualitative evaluation of twelve different beer samples, as shown in table 200b, may include attributes such as, but not limited to, hoppy, fruity, sulfury, bitter, floral, citrus, spicy, sweet, and/or green or grassy, etc. The attributes shown in table 200b are tasting attributes, but other attributes (e.g., non-tasting attributes like color, and/or shelf life) may also be evaluated. The quantitative evaluation, as shown in table 200b, is important for a beer for determining whether the beer meets desired values for one or more attributes. Accordingly, a recipe for brewing a beer is selected to include ingredients and align the manufacturing process for desired values for one or more attributes. In this example of the evaluation of 12 yeasts, the high variability of the results may result in an experimental failure.

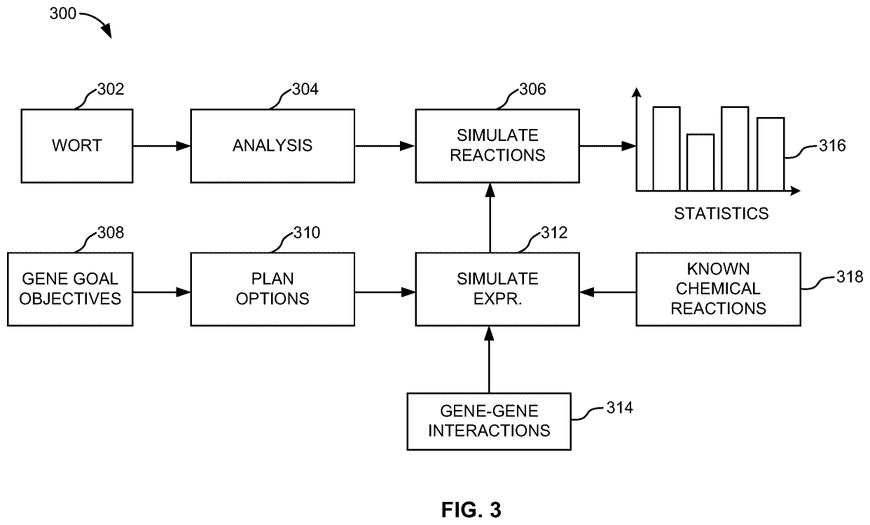

FIG. 3 is an example pipeline 300 to predict an outcome of in-silico fermentation process. In some embodiments, and by way of a non-limiting example, the pipeline 300 assigns signs on interaction relations, for example, upregulate, and/or downregulate, and also uses data or knowledge stored in databases. The pipeline 300 takes control data (e.g., X0 control data) to generate a design matrix, e.g., a design matrix D, that is an output of CRISPR.

In some embodiments, the pipeline 300 takes a wort 302, one or more objectives of optimization (or one or more gene goals) 308, information of known gene-gene interactions 314, and information on known chemical reactions 318, as inputs to generate statistics 316 of a quantity of different compounds that are generated at the end of a fermentation process. The statistics 316 is generated by processing the inputs through analysis 304, option planning 310, simulation of expression 312, and simulation of reactions 306 processing steps, which are described below.

The analysis 304 processing step takes the wort 302 as an input to conduct a process that is conducted using machinery such as, but not limited to only, a spectrometer. An objective of the analysis 304 processing step is to identify a quantity of different compounds that will be consumed during the fermentation process being simulated. Based upon an outcome of the analysis 304 processing step, creation of the wort 302 is adjusted separately. By way of an example, if a precursor of thiols is not found to be present in a predetermined threshold quantity, then a type of grain used and/or a quantity and/or a type of hops used may be adjusted such that the outcome of the analysis 304 processing step yields or generates the outcome that satisfies a predetermined criterion, for example, the precursor of thiols that is at least or exceeds the predetermined threshold quantity.

The option planning 310 processing step takes one or more objectives of optimization (or one or more gene goals) 308 as an input. By way of a non-limiting example, an objective of the one or more objectives of optimization may be over express IRC7 in S. cerevisiae and models to predict what is the best target for the edition. The option planning 310 processing step is performed using services such as, but not limited to, CHOPCHOP that accepts as inputs including, but not limited to, gene names, genomic coordinates, or pasted sequences, which are objectives of optimization or gene goals. Further, CHOPCHOP service then uses off-target search algorithms to predict the specificity of each target site in the genome and generates results that can be provided as an input to the simulation of expression 312 processing step. The simulation of expression 312 processing step, described below, also takes information of known gene-gene interactions 314 and information on known chemical reactions 318 as inputs.

As known to the skilled person, CHOPCHOP is a versatile tool for selecting target sites for CRISPR (Cas9, Cas9 nickase, Cas13, Cpf1/CasX) or TALEN-directed mutagenesis. The V3 release of CHOPCHOP includes many new features and visualization options, supporting over 200 genomes with whole-gene targeting and versatile search modes. The tool now enables precise RNA targeting with Cas13, supporting alternative transcript isoforms and predicting RNA accessibility using ViennaRNA's RNAfold. The update also introduces new DNA targeting modes, including CRISPR activation/repression, targeted enrichment of loci for long-read sequencing, and prediction of Cas9 repair outcomes. For larger queries or handling unsupported genomes, a command-line version is available with additional functionalities. CHOPCHOP V3 is user-friendly and well-equipped to support diverse targeting applications, facilitating effective experimental design.

The simulation of expression 312 processing step disclosed herein takes as input all the possible target sites to simulate expressivity of different genes. As described herein, the actions recommended as an output of the option planning 310 have the desired impact on the target gene in addition to estimating the impact on all of the other genes of the gene pool. By way of a non-limiting example, output of works performed by governmental or non-governmental entities may provide base information that would not be related to a specific gene editing process for a specific context and/or for a specific strain, for example, a specific strain of yeast.

Accordingly, the stimulation of expression 312 processing step uses a specific sequence of the yeast strain edited according to the planned CRISPR operation as an input. The resulting sequence is then scanned to identify the genes in it, and to revise the gene interaction network and estimate the quantities of RNA created during transcription. As a result of the stimulation of expression 312 processing step, the quantity of protein produced during translation would be known.

The simulation of reactions 306 processing step takes as input data from sources such as RHEA (https://www.rhea-db.org/). The output of the simulation of expression 312 processing step and the analysis 304 processing step of the composition of the wort 302 is also used to determine how the components of the wort 302 will react. All this information used to generate a model, e.g., the statistics 316, that will simulate several hours of fermentation process. At every tick of the simulated clock, some compounds will get consumed and some other compounds will be produced. At the end of the simulation process the expected final composition of the chemical solution, for example, a beer, may be produced.

Accordingly, the pipeline 300 to predict an outcome of in-silico fermentation process integrates gene expression data and reaction data. Additionally, the reaction data is combined with gene regulatory network data, and the pipeline 300 provides or predicts a likelihood of different contents or components in a solution.

In some embodiments, data from sources such as RHEA (https://www.rhea-db.org/) is taken as an input. Additionally, an output of the simulation of expression and the analysis of the composition of the wort are also used as inputs as they provide information on how all the components will react. Information corresponding to each of the inputs is combined in a model that simulates several hours of fermentation. At every tick of the simulated clock, some compounds get consumed and some other components are produced. At the end of the simulation process the expected final composition of the chemical solution (e.g., a beer) is produced.

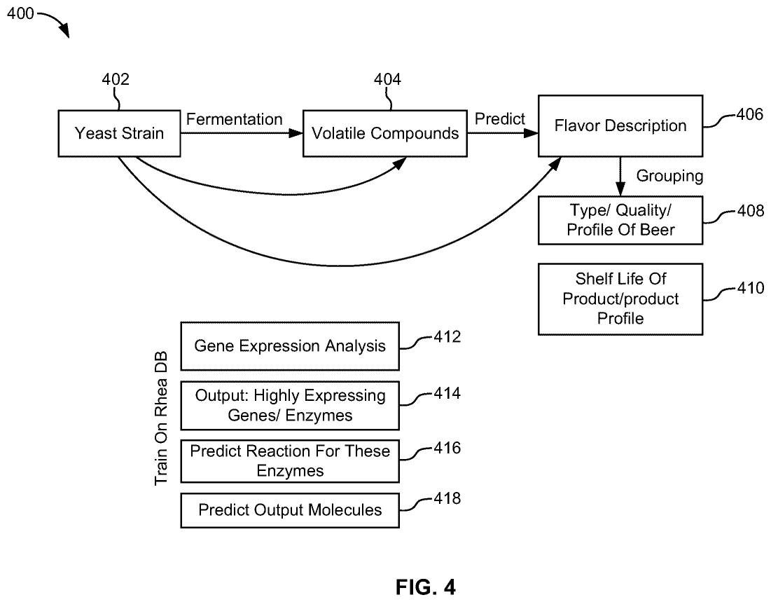

FIG. 4 is an example diagram 400 illustrating an overview of two use-cases derived based upon results shown in tables 200a and 200b, in FIG. 2A and FIG. 2B, respectively, for twelve different yeast strains 402, each yeast strain subjected to a fermentation process. Two sub-objectives (or “use-cases”) that are independent from each other may be identified as (i) prediction of a chemical composition (e.g., volatile compounds) 404 of the produced or manufactured solution; and (ii) prediction of a qualitative feature (e.g., flavor description) 406 of the produced or manufactured solution.

By way of a non-limiting example, the prediction of the chemical composition 404 use-case (referenced herein as a use-case 404) may identify which strain of yeast produces which type of compounds. The use-case 404 includes extracting genes from the yeast strain. The genes may be highly expressed. The use-case 404 further includes identifying genes that are enzymes, and/or identifying types of reactions occurring during the fermentation process and compounds that are generated based upon the types of reactions. The prediction of the qualitative feature 406 use-case (referenced herein as a use-case 406) may identify which strain of yeast produces which flavor of beer. The flavor of beer is identified using compound fingerprinting, for example, using infrared spectroscopy. Additionally, or alternatively, the flavor of beer is determined using polarizable continuum mod (PCM) modeling, which includes compound fingerprinting, for example, using infrared spectroscopy, and yeast gene expression for increasing a number of yeast cells. As shown in FIG. 4, flavor may be determined or predicted based upon the volatile compounds. Based upon the flavor description, a type, quality, and/or a profile of beer, shown in FIG. 4 as 408, and shelf life of the product or product profile, shown in FIG. 4 as 410, may be determined.

As described herein, and as shown in FIG. 4, the PCM modeling may be performed using at least one machine learning algorithm trained using RheaDB, which is an opensource and comprehensive resource of expert-curated biochemical reactions. Further, the use-case 404 and the use-case 406 are two different parts of a pipeline, and, therefore, the use-case 404 and the use-case 406 may be considered independently of each other. By way of a non-limiting example, the at least one machine learning algorithm is trained to perform gene expression analysis 412 to generate an output 414 including information of highly expressing genes or enzymes. Additionally, or alternatively, the at least one machine learning algorithm is trained to predict reactions 416 of the genes or enzymes included in the output 414, and/or predict output molecules 418.

As described herein, a chemical composition of the solution is obtained at the end of the fermentation process, and, accordingly, the chemical composition of the solution depends on number of factors including, but not limited to, genetic composition of the yeast or other fermenting organism, quantity of fermenting agents, external conditions, etc. Currently known technical approaches to predict an output of the fermentation process is based on a normative model. However, yeast, like other organisms, is diverse in nature and exhibits a wide variety across the genetic material. As a result, different yeast strains having different expressivity profiles translate to different flavor and fermentation profiles, for example, for a beer, despite all those different yeast strains are of the same organism, for example, S. cerevisiae.

In some embodiments, and by way of a non-limiting example, PCM modelling, which may be implemented using algorithmic processing such as, simulated annealing or other techniques, takes into account the genetic diversity of yeast, and/or the gene expression data corresponding to each different yeast strain. The gene expression data may be acquired experimentally or based upon an output of a prediction taking into account the gene editing process described herein.

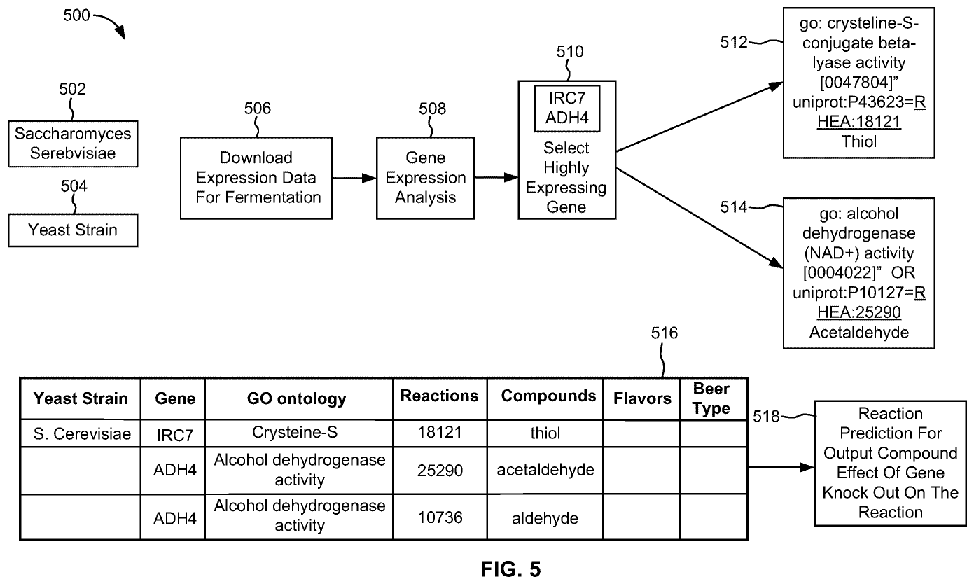

The gene expression analysis 412 is performed for the use-case 404 and is described in detail with regards to a flow-diagram 500 shown in FIG. 5. As shown in the flow-diagram 500, expression data for fermentation is downloaded 506 for a yeast organism 502, and different strains 504 of the yeast organism 502. The expression data for fermentation may be downloaded 506 from Saccharomyces Genome Database (SGD), SPELL, YeasyNet, and/or UCSC Genome Browser Gateway, etc.

Based upon the downloaded 506 expression data, gene expression analysis 508 is performed. The gene expression analysis 508 identifies a pattern of genes expressed at the level of genetic transcription under specific circumstances or in a specific cell of the yeast organism 502 and/or different strains 504. The gene expression analysis 508 may be performed using one or more technologies including, but not limited to, real time quantitative RT-PCR, in situ hybridization, microarrays, and/or massively parallel signature sequencing (MPSS), etc. The gene expression analysis 508 predicts a list of highly expressive genes and/or enzymes 510. For one or more of expressive genes and/or enzymes included in the list of highly expressive genes and/or enzymes 510, reaction prediction 512 and compound prediction 514 are performed using RheaDB. By way of an example, the reaction prediction 512 and/or the compound prediction 514 may be in a matrix form similar to table 200b and/or table 200a, shown in FIG. 2B and FIG. 2A, respectively. In other words, the compound prediction 514 predicts what kind of compound will be found in the finished product and in which respective quantity. The reaction prediction 512 and/or the compound prediction 514 are made taking into account the gene expression data for the yeast organism 502 and/or different strains 504, in addition to the chemical composition of the fermenting liquid used at the start of the fermentation process. In FIG. 5, information included in a table 516 for a yeast strain S. cerevisiae and genes (for example, IRC7 and ADH4) and compounds such as, thiol, acetaldehyde, and/or aldehyde, etc., is used for reaction prediction 518 for outputting or predicting compound effect of gene knock out on the reaction.

Beyond the chemical composition or the use-case 404, assessing a range of phenotypic qualities of the product produced using the fermentation process is another important aspect, for example, the use-case 406. The range of phenotypic qualities may include, but not only limited to, a color, and/or a shelf-life of the product produced using the fermentation process.

As shown in the table 200b, the results are subjective to some extent, and therefore a simple approach of direct, deterministic, association between the presence of a compound and some qualifiers may not be efficient to use. Also, the quantity of the compounds, e.g., a list of compounds shown in table 200a, plays a role to the final assessment. Accordingly, the final assessment cannot be simply made using a simple Boolean flag for presence or absence of a specific compound.

Lastly, some compounds may be different in denomination but may still lead to the same final assessment. Such compounds are referenced or identified as biosimilar active substances. By way of an example, in table 200a the items in rows 8 and 10 may behave the same in terms of their impact on the labels associated to the product. In the embodiments described herein, labels may be predicted based upon fingerprinting of compounds, where the fingerprinting of a compound is associated or corresponds with a number.

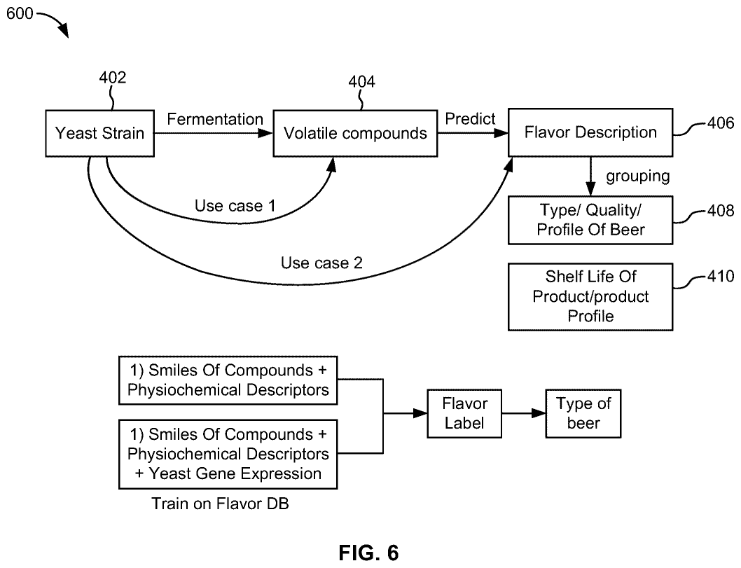

The use-case 406, which is to determine or predict which strain of yeast would produce which flavor is independent from the use-case 404. However, the use-case 406 may be performed taking into account an outcome of the use-case 404. Prediction for the use-case 406 is performed using compound fingerprinting and/or the PCM modeling as shown in a flow-diagram 600 shown in FIG. 6.

As shown in the flow-diagram 600, compound fingerprints to predict flavors may be performed using a Simplified Molecular Input Line Entry System (SMILES) method that is a linear notation method and uses a fixed alphabet to represent chemical compounds as strings, referenced herein as SMILES strings. SMILE strings are a two-dimensional picture of a molecule and use specific characters and grammar to describe the structure and atoms of a chemical compound. Further, SMILES strings express structural differences, such as chirality. Additionally, compound fingerprints to predict flavors may be based upon physiochemical descriptors.

As described herein, the PCM modeling includes compound fingerprints and yeast gene expression analysis using a machine learning algorithm trained using flavorDB, which is resource with extensive coverage of about 25,595 flavor molecules.

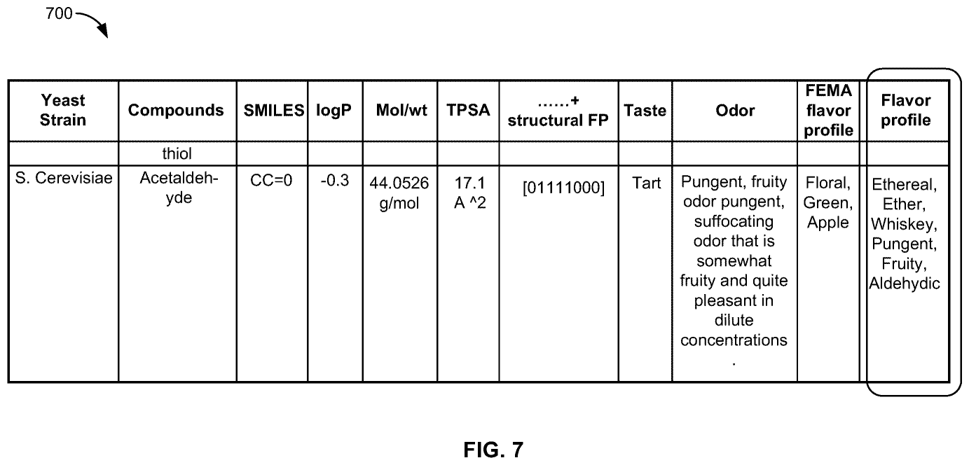

FIG. 7 is an example flavor profile 700 generated using compound fingerprinting shown in FIG. 6. As shown in FIG. 7, for yeast strain S. cerevisiae with compounds acetaldehyde and SMILES spring CC=O, attributes such as, taste, odor, FEMA flavor profile, and/or flavor profile may be determined or predicted.

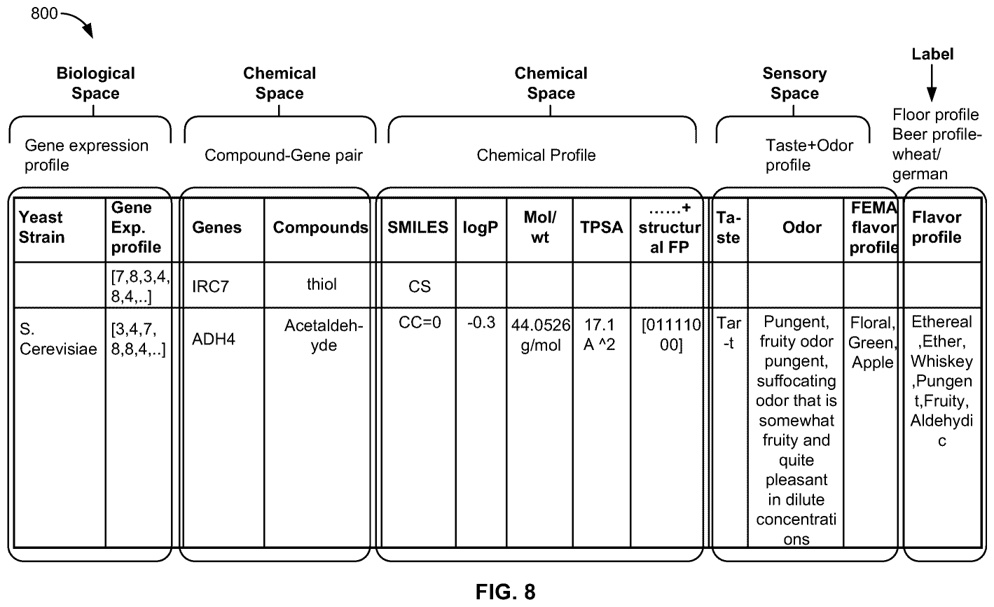

FIG. 8 is an example flavor profile 800 generated using the PCM modeling. As shown in FIG. 8, for yeast strain S. cerevisiae, a gene expression profile (or biological space), and a chemical profile (or chemical space) corresponding to compounds and/or genes may be determined or predicted. Additionally, attributes (or sensory space) including taste, odor, and/or flavor extracts manufactures association (FEMA) flavor profile, and flavor profile (shown in FIG. 6 as flavor label) may be determined or predicted. Based upon the flavor label, a type of beer may be determined or predicted.

In FIG. 7 and FIG. 8, one of the compounds generated in the solution from the fermentation process is Thiols, which are a family of compounds giving aromatic notes of tropical fruits to the beer. These flavors are sought after for the beer styles called IPA and NEIPA. Brewers making these kinds of beers have interests in maximizing the amount of Thiols and this could be achieved by either changing the mash (the grains used for fermentation), modifying the hops used (type and/or quantity) or tweaking the yeast. Among those three options, tweaking the yeast is the most cost-effective option as it does not require tuning the inputs of the process.

Thiols are generated via an enzyme performing what is called a beta-lyase activity on pre-cursor compounds. The enzyme essentially consumes pre-cursor compounds and turn them into Thiols. The gene IRC7 has been identified as encoding for this enzyme but is typically not found or not active enough in most strains of S. cerevisiae yeast organism that is used for beer brewing. Accordingly, CRISPR/Cas9 is used to either insert or over-express the gene IRC7 in it.

However, inserting or over-expressing the gene IRC7 is challenging to perform only that change without modifying other components or activities of the yeast. Even though yeast is a mono-cellular organism, it nonetheless performs many activities across its 6000 or so coding and non-coding genes. Currently known approach using CRISPR intervention is costly in both time and resources. Further, the focus on potential outcome for gene editing also misses on account for the impact of gene-gene interaction in the cell because another gene may temper its expressivity of the gene IRC7.

Accordingly, embodiments in the present disclosure describe leveraging artificial intelligence (AI) based techniques to predict in-silico the most likely outcomes of wet lab experiments. Using the AI based techniques makes it possible to save resources by prioritizing the executions of the approaches that are most likely to lead to the desired end result.

During beer production, yeast cells, typically Saccharomyces cerevisiae, are alive and active during the fermentation process. Yeast cells consume sugars in the wort and convert them into alcohol and carbon dioxide through anaerobic respiration. This metabolic activity is crucial for alcohol production and contributes to the characteristic flavors and aromas in beer. Yeast cells undergo various metabolic processes including sugar metabolism, alcohol production, and the release of flavor compounds. Yeast cells also undergo changes in gene expression and regulatory processes to adapt to the fermentation environment and fulfill their role in beer production.

As described herein, the gene expression patterns and aging distribution of yeast cells during the fermentation process may be influenced by multiple factors including, but not only limited to, yeast strain characteristics, fermentation conditions (such as temperature and nutrient availability), the specific brewing process, temperature, pH, nutrient levels, and/or rate of fermentation. By way of an example, these factors impact the gene regulatory network (GRN) and gene expression patterns of yeast cells during the fermentation process.

In the embodiments disclosed herein, the GRN may be modeled being a main indicator for the final expression. By way of a non-limiting example, the microenvironment (e.g., temperature, pH, and nutrient levels) and other factors may be modeled separately into a phase known as a reaction phase. During the reaction phase, the gene expression patterns and potential interactions of yeast cells without incorporating the complex dynamics of the fermentation microenvironment are considered. In other words, the embodiments predict a GRN following a target edition (e.g., up-regulate a particular gene) and an outcome of a differential gene expression analysis following the target edition such that different tests can be rapidly iterated until the target result is achieved saving time and resources.

In some embodiments, and by way of a non-limiting example, a system for increasing expressivity of the gene IRC7 may take the standard GRN of a non-modified yeast strain (X0) and a target modification (D) as inputs to produce a matrix and a modified GRN as outputs. The matrix may include a gene and a level of expression to be used for reaction data.

In some embodiments, to increase IRC7 in a yeast, X0 may be the normal GRN for the yeast, wherein this data may be obtained publicly or figured out in a lab, and D is a matrix indicating an increase of IRC7 across all the cells. The output may be a table indicating an increased expression of IRC7 and/or an unanticipated over-expression of a different gene up-regulated in a complex way across the new GRN (X).

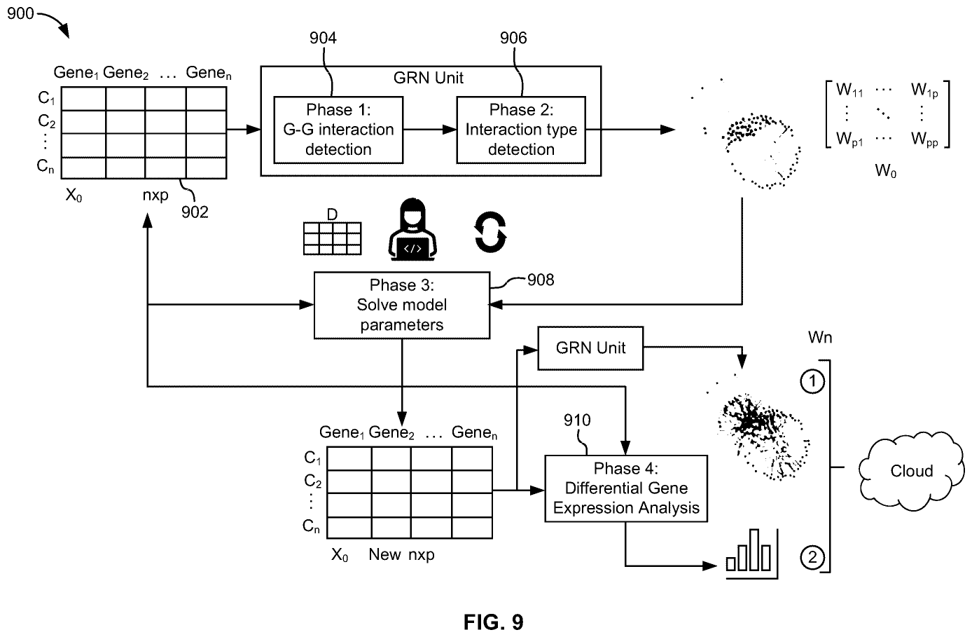

FIG. 9 is an example flow-chart 900 of the steps involved in up-regulation of the IRC7 gene-expression. As shown in the flow-chart 900, C₁, C₂, . . . Cₙ represent a set of yeast cells (or a population of yeast cells). In some embodiments, and by way of a non-limiting example, for log-scale normalized gene expression values for n initial yeast cells (a heterogeneous population of yeast cells) across p genes organized in an n×p expression matrix X 902, as shown in the flow-chart 900, there may be following four phases: (i) GRN inferring from expression matrix phase 904 (or phase-1 904); (ii) gene-gene casualty inferring phase 906 (or phase-2 906); (iii) applying in-silico modification on the target gene and predicting new expression matrix and consequent regulatory network for the next steady state phase 908 (or phase-3 908); and (iv) performing differential gene expression analysis between two matrices X and X0 phase 910 (or phase-4 910), each of which is described in detail below.

In some embodiments, during the phase-1 904, an initial GRN of the set of yeast cells C₁, C₂, . . . Cₙ is inferred using a tree-based machine learning algorithm. By way of an example, the tree-based machine learning algorithm may be a random forest regression model, which is trained to predict the expression level of a target gene xi based on the expression levels of all other genes (x₁, x₂, . . . , xᵢ₋₁, xᵢ, xᵢ₊₁, . . . xₙ)T, in the dataset. The random forest tree is constructed recursively by splitting the data into smaller subsets based on the expression levels of a randomly selected subset of genes. The splitting process continues until a stopping criterion is met, such as reaching a maximum depth or a minimum number of cells in each leaf. In one example, the minimum cell count in each leaf may be set to 10 to ensure a greater likelihood of accurately representing rare cell populations.

During the phase-2 906, information about the types of interactions is inferred. Interactions between genes can occur in various forms. Additive interactions involve the cumulative or combined effect of multiple genes, which is the sum of individual gene effects. Multiplicative or synergistic interactions result in a combined effect that is the product of individual gene effects. Suppressive or epistatic interactions occur when one gene masks the effect of another. Complementary interactions involve genes working together to control a phenotype, and the absence of any single gene alters the phenotype. Modifier interactions happen when one gene modifies the effect of another.

During the phase-3 908, influence of perturbations on gene expression is assessed using a computational framework employing a regularized linear model. The linear model predicts expression levels of individual genes (represented by the expression matrix X) based upon the combined effects of guide molecules (represented by the design matrix D) and adjacency matrix of initial regulatory network GRNcs, WO, on the initial expression matrix X0.

During the phase-4 910, gene expression changes between X and X0 are examined by performing a differential gene expression analysis. By way of an example, differential expression is examined by performing the non-parametric Kruskal-Wallis test, in which the significant value of p-value indicates that the gene expression in at least one state has a higher rank than another one based on stochastic dominance. However, because calculating differential expression may introduce a bias in the distribution of p-values, the p-values are used for ranking the genes primarily. An output of the phase-4 910 includes a list of genes with corresponding changes rate in their expression. Additionally, or alternatively, the output of the phase-4 910 further includes a matrix (Wn) including two columns, for example, gene, and level of expression. The matrix Wn may be a table indicating an increased expression of IRC7. The increased expression of IRC7 may be as desired or an unanticipated over-expression of a different gene up-regulated in a complex way across the new GRN (X).

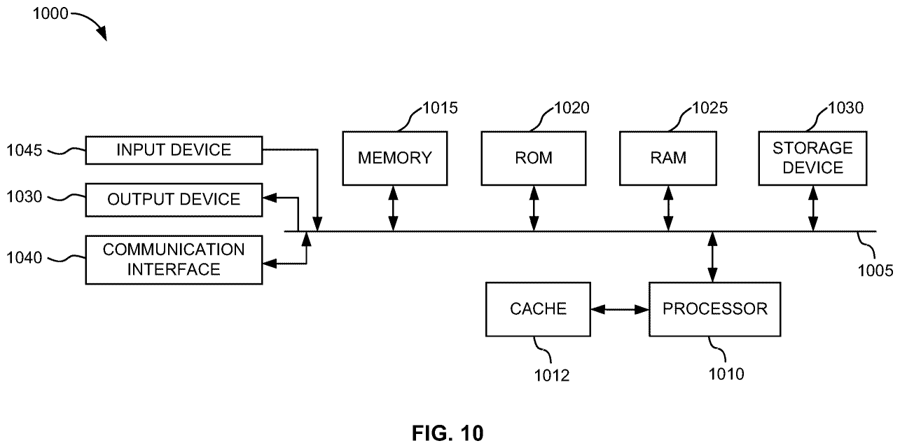

FIG. 10 illustrates an example computing system 1000 that can implement various techniques, processes, functions, or methods described herein. The components of computing system 1000 are shown in electrical communication with each other using a connection 1005, such as a bus. The example computing system 1000 includes a processing unit (CPU or processor) 1010 and a computing device connection 1005 that couples various computing device components, including computing device memory 1015, such as a read only memory (ROM) 1020 and a random-access memory (RAM) 1025, to processor 1010.

Computing system 1000 can include a cache 1012 of high-speed memory connected directly with, in close proximity to, or integrated as part of processor 1010. Computing system 1000 can copy data from memory 1015 and/or storage device 1030 to cache 1012 for quick access by processor 1010. In this way, cache 1012 can provide a performance boost that avoids processor 1010 delays while waiting for data. These and other modules can control or be configured to control processor 1010 to perform various actions. Other computing device memory 1015 may be available for use as well. Memory 1015 can include multiple different types of memory with different performance characteristics. Processor 1010 can include any general-purpose processor, central processing unit (CPU), or graphics processing unit (GPU) in combination with a hardware or software provision configured to control processor 1010 and stored in storage device 1030, as well as any special-purpose processor where software instructions are incorporated into the processor design. Processor 1010 may be a self-contained system, containing multiple cores or processors, a bus, memory controller, cache, etc. A multi-core processor may be symmetric or asymmetric.

Storage device 1030 is a non-volatile memory and can be one or more of a hard disk or other types of computer readable media that can store data that are accessible by a computer, such as a magnetic cassette, flash memory card, solid state memory device, digital versatile disk, cartridge, RAM 1025, ROM 1020, or hybrids thereof. Memory 1015 or storage device 1030 can include software, code, firmware, etc., for controlling processor 1010. Other hardware or software modules are contemplated. Memory 1015 and storage device 1030 are connected to computing device connection 1005. In one aspect, a hardware module that performs a particular function can include the software component stored in a computer-readable medium in connection with the necessary hardware components, such as processor 1010, computing device connection 1005, and so forth, to carry out the function. In the example embodiment, processor 1010 may be programmed by encoding an operation or function using one or more executable instructions and providing the executable instructions in memory 1015 or storage device 1030.

In operation, a computer executes computer-executable instructions embodied in one or more computer-executable components stored on one or more computer-readable media to implement aspects of the disclosure described or illustrated herein. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

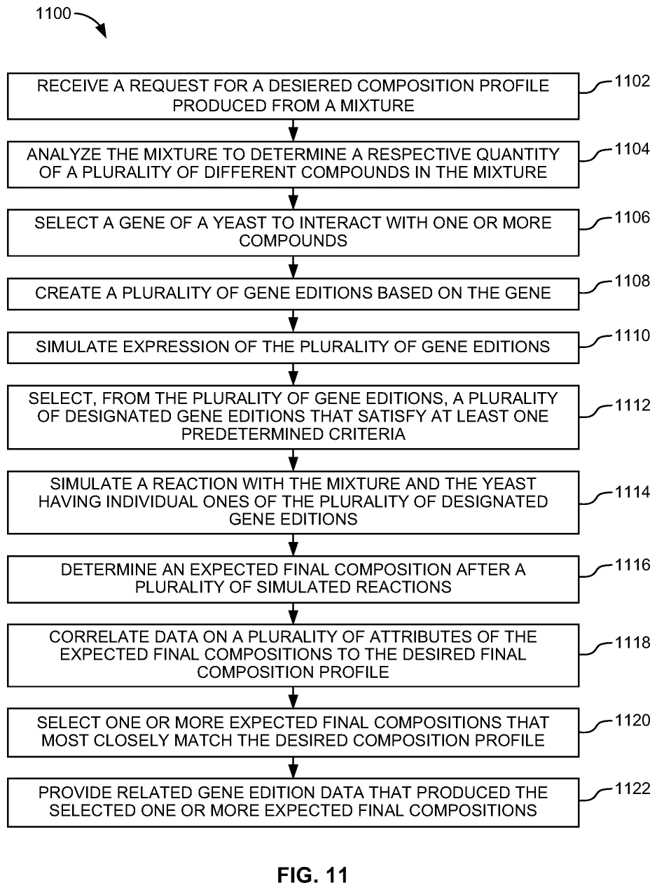

FIG. 11 is an example flow-chart 1100 of method operations for selecting an engineered organism with a desired protein expression. The method operations may be performed by a computing device shown in FIG. 10. The computing device shown in FIG. 10 may be a personal computing device, an application server, a laptop, a tables, and/or any electronic computing device. The method operations may include receiving 1102 a request for a desired composition profile produced from a mixture. The request may be received as an input from a user of the computing device 1000 via the input device 1045 and/or the communication interface 1040. The desired composition profile may include, but not limited to, a desired flavor, a desired type, a desired quality, a desired profile of a beer, a desired shelf life of a product being produced using a fermentation process.

The method operations may include analyzing 1104 the mixture to determine a respective quantity of a plurality of different compounds in the mixture. In particular, analyzing 1104 the mixture to determine the respective quantity of the plurality of different compounds may be performed as described herein with reference to FIG. 4. The respective quantity of the plurality of different compounds may be as shown in table 200a.

The method operations may include selecting 1106 a gene of a yeast to interact with one or more of the compounds. As described herein, for a yeast strain S. cerevisiae, the selected gene of the yeast may include, but not limited to, IRC7, and/or ADH4. The selected gene may be selected for its interaction with one or more compounds such as, thiol, acetaldehyde, and/or aldehyde, etc.

The method operations may include creating 1108 a plurality of gene editions based on the gene and simulating 1110 expression of the plurality of gene editions to determine impact on the gene and a plurality of other genes. In one example, the plurality of gene editions may be created, and the expression of the plurality of gene editions may be simulated, according to the stimulation of expression 312, as described herein. Accordingly, details of these operations are not repeated for brevity.

The method operations may include selecting 1112 a plurality of designated gene editions that satisfy at least one predetermined criteria. The plurality of designated gene editions are selected from the plurality of gene editions, The method operations may include simulating 1114 a reaction with the mixture and the yeast having individual ones of the plurality of designated gene editions. The simulating 1114 includes a plurality of hours, the mixture, and the yeast having the individual ones of the plurality of designated gene editions. The method operations may include determining 1116 an expected final composition after a plurality of the simulated reactions, and correlating 1118 data on a plurality of attributes of the expected final compositions to the desired final composition profile. Since details of these operations are described above in detail, those details are not repeated again for sake of brevity.

The method operations may include selecting 1120 one or more expected final compositions that most closely match the desired composition profile, and providing 1122 related gene edition data that produced the selected one or more expected final compositions, as described in detail above.

An example technical effect of the methods, systems, and apparatus described herein includes at least predict in-silico, by leveraging artificial intelligence (AI) based techniques, the most likely outcomes of wet lab experiments. Further, using the AI based techniques makes it possible to save resources by prioritizing the executions of the approaches that are most likely to lead to the desired end result.

Various embodiments or aspects disclosed herein thus comprise a method of selecting an engineered organism with a desired protein expression, the method comprising: (i) receiving a request for a desired composition profile produced from a mixture; (ii) analyzing the mixture to determine a respective quantity of a plurality of different compounds in the mixture; (iii) selecting a gene of a yeast to interact with one or more of the compounds; (iv) creating a plurality of gene editions based on the gene; (v) simulating expression of the plurality of gene editions to determine impact on the gene and a plurality of other genes; (vi) selecting, from the plurality of gene editions, a plurality of designated gene editions that satisfy at least one predetermined criteria; (vii) simulating a reaction with the mixture and the yeast having individual ones of the plurality of designated gene editions, wherein the simulating includes a plurality of hours, the mixture, and the yeast having the individual ones of the plurality of designated gene editions; (viii) determining an expected final composition after a plurality of the simulated reactions; (ix) correlating data on a plurality of attributes of the expected final compositions to the desired final composition profile; (x) selecting one or more expected final compositions that most closely match the desired composition profile; and (xi) providing related gene edition data that produced the selected one or more expected final compositions.

The method according to embodiments or aspects described above, wherein the mixture is a wort for brewing a beer.

The method according to any embodiment or aspect described above, wherein the plurality of attributes includes one or more of hoppy, fruity, sulfury, bitter, floral, citrus, green, spicy, and/or sweet.

The method according to any embodiment or aspect described above, wherein the simulating the expression of the plurality of gene editions comprises inferring, based upon a tree-based machine learning algorithm, an initial granulin precursor or an initial metabolite precursor of n number of initial yeast cells.

The method according to any embodiment or aspect described above, wherein the tree-based machine learning algorithm includes a random forest regression model trained to predict an expression level of the gene based on corresponding expression levels of all other genes in the data set.

The method according to any embodiment or aspect described above, wherein the tree-based machine learning algorithm is constructed by recursively splitting data into smaller subsets based on expression levels of a randomly selected subset of genes until a predetermined criteria is satisfied.

The method according to any embodiment or aspect described above, further comprising analyzing gene interactions to determine a combined effect including one or more of a synergistic interaction, complementary interaction, and/or modifier interaction.

The method according to any embodiment or aspect described above, further comprising executing a regularized linear model to determine influence of perturbations on gene expression, wherein the model predicts, based on at least one combined effect of guide molecules and/or initial regulatory network granulin precursors, expression levels of the gene and the all other genes.

The method according to any embodiment or aspect described above, wherein the mixture is feedstock for a synthetic biology production.

The method according to any embodiment or aspect described above, wherein the plurality of different compounds in the mixture comprise acetaldehyde (CAS No. 75-07-0), dimethyl sulfide (CAS No. 75-18-3), ethyl acetate (CAS No. 141-78-6), ethanol (CAS No. 64-17-5), ethyl propanoate (CAS No. 105-37-3), ethyl 2-methylpropanoate (CAS No. 97-62-1), n-propyl acetate (CAS No. 109-60-4), 4-methyl-2-pentanone (CAS No. 108-10-1), 2-methylpropyl acetate (CAS No. 110-19-0), 3-methyl-2-pentanone (CAS No. 565-61-7), α-pinene (CAS No. 80-56-8), or any combination thereof.

The method according to any embodiment or aspect described above, further comprising validating the selection of the engineered organism.

The method according to any embodiment or aspect described above, wherein validating the selection of the engineered organism comprises measuring the expression of one or more genes of the engineered organism in a fermenting liquid or a fermented product.

The method according to any embodiment or aspect described above, further comprising repeating the measuring the expression of one or more genes of the engineered organism in a fermenting liquid.

The method according to any embodiment or aspect described above, wherein measuring gene expression can comprise using high-density expression array, DNA microarray, polymerase chain reaction (PCR), reverse transcriptase PCR (RT-PCR), real-time quantitative reverse transcription PCR (qRT-PCR), serial analysis of gene expression (SAGE), spotted cDNA arrays, GeneChip, spotted oligo arrays, bead arrays, RNA Seq, tiling array, northern blotting, hybridization microarray, in situ hybridization, whole-exome sequencing, whole-genome sequencing, liquid biopsy, next-generation sequencing, or any combination thereof.

The method according to any embodiment or aspect described above, wherein validating the selection of the engineered organism comprises measuring the reaction data.

The method according to any embodiment or aspect described above, wherein measuring reaction data comprises analyzing the chemical composition of fermenting liquid or fermented product.

The method according to any embodiment or aspect described above, wherein measuring reaction data comprises analyzing the flavor composition of fermenting liquid or fermented product.

The method according to any embodiment or aspect described above, wherein validating the selection of the engineered organism comprises building a network of gene regulation data.

The method according to any embodiment or aspect described above, further comprising identifying gene regulation data associated with a desired flavor profile.

The method according to any embodiment or aspect described above, further comprising predicting the flavor profile based on chemical composition data.

The method according to any embodiment or aspect described above, further comprising predicting the flavor profile based on gene expression data.

The method according to any embodiment or aspect described above, further comprising predicting the flavor profile based on sensory data.

The method according to any embodiment or aspect described above, wherein chemical composition data, gene expression data, and/or sensory data predicts the flavor profile of fermenting liquid or fermented product.

The method according to any embodiment or aspect described above, wherein validating the selection of the engineered organism comprises identifying the one or more gene editions that provide the desired flavor profile.

In an aspect, numerous bacterial nucleic acid modification systems have led to the recent development of two modular, precise genome editing tools. The TALE (transcription activator-like effector) and CRISPR/Cas (clustered regularly interspaced short palindromic repeats) systems have recently been optimized for research use to site-specifically introduce mutations and manipulate transcriptional activation and repression in a variety of organisms.

TALENs are a genome editing method derived from plant pathogenic bacteria. TALE architecture is composed of three parts: an N-terminal domain, TALE repeat domains, and a C-terminal domain. The TALE repeat domains typically consist of 34 amino acid residues, where the 12th and 13th repeat variable di-residues (RVDs) determine DNA nucleotide binding specificity. Each RVD recognizes a specific nucleotide, leading to a simple code for DNA recognition: NI for adenine, HD for cytosine, NG for thymine and NH or NN for guanine. Importantly, the RVDs can be assembled sequentially to bind any given target sequence. For genome editing purposes, TALEs are fused to the FokI nuclease domain to create TALE nucleases (TALENs). Because FokI only cleaves as a dimer, sites must be targeted by a pair of TALENs binding on opposite faces of the DNA strand, spaced ˜14-20 bp apart. The FokI nuclease domains dimerize across the spacer sequence and create a double-strand break (DSB). The DSB can be repaired through error-prone non-homologous end-joining (NHEJ), which often results in indels and potentially frameshift mutations. For efficient binding, TALEN target sequences require a thymine at the 5′ end for recognition by the TALE N-terminus.

The CRISPR/Cas9 system originates from a bacterial immune system that has been adopted for use as a programmable genome editing tool. Streptococcus pyogenes Cas9 nuclease is directed to target sites in the genome by a single-guide RNA (sgRNA). The Cas9/sgRNA complex binds a 20 bp target sequence followed by a 3 bp protospacer adjacent motif (PAM)-NGG (two invariable Gs preceded by a variable base), and it creates a DSB that is repaired in a seemingly identical manner to TALEN-induced DSBs. While the presence of an -NGG PAM motif is one of the few requirements for binding, the methods used to generate sgRNAs for targeting often impose additional restrictions. Depending on the polymerase used for sgRNA synthesis, the 5′ end dinucleotides can be limited to, for example, 5′ GN- for the commonly used U6 promoter (polymerase III), or 5′ GG- for T7 polymerase. In addition, certain criteria such as guanine-cytosine content (GC-content) appear to influence binding efficiency. These, along with other guidelines to ensure target suitability, have been used to mostly manually design sgRNAs to generate mutations and knockouts in a variety of organisms including bacteria, yeast, zebrafish, Xenopus, nematodes, fruit flies, mice and human cells.

TALEN and sgRNA design require identification of target sites that fulfill certain sequence requirements while simultaneously avoiding off-targets elsewhere in the genome. Several studies have demonstrated the limited specificity of TALEN- and particularly Cas9-based genome editing strategies, highlighting the importance of determining the uniqueness of each candidate target site.

Existing tools for identifying TALEN or sgRNA target sites have limitations, including acceptance of few input formats, slow search times, restriction to either TALEN or CRISPR/Cas9 target design, minimal or no visualization of the target locus and/or limited information about potential off-target sites.

The method according to any embodiment or aspect described above, wherein the method optimizes an engineered organism for fermentation.

The method according to any embodiment or aspect described above, wherein validating the selection of the engineered organism comprises identifying the one or more gene editions that provide the desired flavor profile.

The method according to any embodiment or aspect described above, wherein the desired flavor profile is that of an Standard American Beer, International Lager, Czech Lager, Pale Malty European Lager, Pale Bitter European Beer, Amber Malty European Lager, Amber Bitter European Beer, Dark European Lager, Strong European Beer, German Wheat Beer, British Bitter, Pale Commonwealth Beer, Brown British Beer, Scottish Ale, Irish Beer, Dark British Beer, Strong British Ale, Pale American Ale, Amber And Brown American Beer, American Porter And Stout, Strong American Ale, European Sour Ale, Belgian Ale, Strong Belgian Ale, Monastic Ale, Historical Beer, American Wild Ale, Spiced Beer, Smoked Beer, Specialty Beer, Argentine Styles (e.g., Dorada Pampeana, IPA Argenta, etc.), Italian Styles (e.g., Italian Grape Ale), Brazilian Styles (e.g., Catharina Sour), New Zealand Styles (e.g., New Zealand Pilsner).

The method according to any embodiment or aspect described above, wherein the desired flavor profile considers Ethyl Acetate (EA), Ethanol, 1-Propanol (1P), 1-Propanol, 2-methyl-(1P2M), 1-Butanol, 3-Methyl-(1B3M), Ethyl Ester Octanoic Acid (OCE), 2-Oxo-Propanoic Acid (2PA), 2-Phenylethyl Acetate (2-PEAC), Hexanoic acid (HA), Phenylethyl Alcohol (PA), 1,2-Benzisothiazole (12B), Octanoic Acid (OA), n-Decanoic acid (nDC), 2,4-Di-tert-butylphenol (24DTB), 9-Decenoic Acid (9DC), Acetaldehyde, acetoxy-3-methoxycinnamaldehyde (4A3M), 1-Butanol, 3-methyl-, acetate (IA), 1-Butanol, 2-methyl-, acetate (AAA), Decanoic acid, ethyl ester (DCE), Hexanoic acid, ethyl ester (HCE), Oxalic Acid, Cyclobutyl Hexadecyl Ester (OCHE), Butanoic acid, butyl ester (BB), 2-Ethyl-Heptanoic Acid (2EHC), 1-Heptanol, 2-Propyn-1-ol (2PP), or any combination thereof.

The method according to any embodiment or aspect described above, further comprising introducing one or more gene editions into a native or wild-type organism to create an engineered organism.

The method according to any embodiment or aspect described above, wherein introducing one or more gene editions comprises using a gene editing system.

The method according to any embodiment or aspect described above, wherein the gene editing system comprises using zinc-finger nucleases (ZFNs), transcription activator-like effector nucleases (TALENs), meganucleases, or clustered regularly interspaced short palindromic repeats (CRISPR).

The method according to any embodiment or aspect described above, wherein CRISPR comprises CRISPR/Cas9.

The method according to any embodiment or aspect described above, wherein the gene editions modulate the expression level of one or more genes.

The method according to any embodiment or aspect described above, wherein the gene editions modulate the expression level of one or more genes involved in fermentation.

The method according to any embodiment or aspect described above, wherein the gene editions modulate the expression level of one or more genes involved in one or more biological processes.

In an aspect, modulating can comprise decreasing and/or reducing expression and/or activity of the target gene or a portion of the target gene.

In an aspect, modulating can comprise increasing and/or enhancing expression and/or activity of the target gene or a portion of the target gene.

For example, in an aspect, a disclosed method can comprise modulating the expression and/or activity of ADH1; ADH2; ADH3; ADH4; ADH5; ADH6; ADH7; ALD; ARO10; BAT1; BAT2; CHA1; EHT1; FAA4; HOR7; ILV3; ILV5; ILV6; IMA1; IRA1; IRC7; LEU1; LEU2; LEU4; LEU9; QCR7; OLE1; PDC; PUT1; SFA1; SSD1, THI3; TPS2; or any combination thereof.

The method according to any embodiment or aspect described above, wherein modulate comprises increasing the expression level of one or more genes, or wherein modulating comprises decreasing the expression of one or more genes.

The method according to any embodiment or aspect described above, wherein modulate comprises increasing the expression level of one or more genes and decreasing the expression of one or more genes.

The method according to any embodiment or aspect described above, wherein the one or more biological processes comprise peptide biosynthesis, cellular amide metabolism, amide biosynthesis, peptide metabolism, organonitrogen compound biosynthesis, biosynthesis, macromolecule biosynthesis, organic substance biosynthesis, cellular macromolecule biosynthesis, cellular biosynthesis, cellular protein metabolism, gene expression, cellular nitrogen compound biosynthesis, protein metabolism, organonitrogen compound metabolism, response to oxidative stress, anion transport, cellular macromolecule metabolism, aspartate family amino acid metabolism, carboxylic acid biosynthesis, organic acid biosynthesis, branched-chain amino acid metabolism, branched-chain amino acid biosynthesis, sulfur amino acid metabolism, fatty acid biosynthesis, monocarboxylic acid biosynthesis, small molecule biosynthesis, fatty acid metabolism, sulfur amino acid biosynthesis, energy derivation by oxidation of organic compounds, or any combinations thereof.

The method according to any embodiment or aspect described above, wherein the one or more gene editions affect ADH1-ADH7, (alcohol dehydrogenase); ALD (alanine dehydrogenase); ARO10 (phenylpyruvate decarboxylase); BAT1 (branched-chain-amino-acid); transaminase); BAT2 (branched-chain-amino-acid transaminase); CHA1 (1-serine/1-threonine ammonia-lyase); EHT1 (medium-chain fatty acid ethyl ester synthase/esterase), FAA4 (fatty acid activation 4); HOR7; ILV3 (dihydroxy-acid dehydratase); ILV5 (ketol-acid reductoisomerase); ILV6 (acetolactate synthase regulatory subunit); IMA1 (isomaltase); IRA1 (GTPase-activating protein); IRC7; LEU1 (3-isopropylmalate dehydratase); LEU2 (3-isopropylmalate dehydrogenase); LEU4 (2-isopropylmalate synthase); LEU9 (2-isopropylmalate synthase); QCR7 (ubiquinol-cytochrome C oxidoreductase); OLE1 (oleic acid requiring); PDC (pyruvate decarboxylase); PUT1 (proline utilization); SFA1 (bifunctional alcohol dehydrogenase), SSD1, THI3 (branched-chain-2-oxoacid decarboxylase); TPS2 (Trehalose-6-Phosphate Synthase/phosphatase); or any combination thereof.

The method according to any embodiment or aspect described above, wherein the engineered organism is yeast.

The method according to any embodiment or aspect described above, wherein the engineered organism is Saccharomyces cerevisiae or Saccharomyces pastorianus.

In some embodiments, the system may be configured to implement machine learning models, including neural network, that “learns” to analyze, organize, process, and/or validate data without being explicitly programmed. Machine learning may be implemented through machine learning (ML) methods and algorithms. In an exemplary embodiment, a machine learning (ML) module; may be configured to implement ML methods and algorithms. In some embodiments, ML methods and algorithms may be applied to data inputs and generate machine learning (ML) outputs. Data inputs may include but are not limited to: analog and digital signals, sensor data, image data, video data, datasets stored in one or more databases, and the like. ML outputs may include but are not limited to: digital signals, matrices, predictions and guidance, and the like. In some embodiments, data inputs may include certain ML outputs.

In some embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, recurrent neural networks, Monte Carlo search trees, generative adversarial networks, dimensionality reduction, and support vector machines. In various embodiments, the implemented ML methods and algorithms may be directed toward at least one of a plurality of categorizations of machine learning, such as supervised learning, unsupervised learning, and reinforcement learning.

In some embodiments, ML methods and algorithms may be directed toward supervised learning, which involves identifying patterns in existing data to make predictions about subsequently received data. Specifically, NIL methods and algorithms directed toward supervised learning are “trained” through training data, which includes example inputs and associated example outputs. Based on the training data, the IL methods and algorithms may generate a predictive function which maps outputs to inputs and utilize the predictive function to generate ML outputs based on data inputs. The example inputs and example outputs of the training data may include any of the data inputs or ML outputs described above. For example, a ML module may receive training data comprising data associated with different patients and their corresponding outcomes, generate a model which maps the patient data to the outcome data, and recognize potential future outcomes for patients.

In some embodiments, MIL methods and algorithms may be directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In an exemplary embodiment, a NIL module coupled to or in communication with the design system or integrated as a component of the design system receives unlabeled data, and the ML module employs an unsupervised learning method such as “clustering” to identify patterns and organize the unlabeled data into meaningful groups. The newly organized data may be used, for example, to extract further information about the potential classifications.

In some embodiments, NIL methods and algorithms may be directed toward reinforcement learning, which involves optimizing outputs based on feedback from a reward signal. Specifically, ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated MIL outputs. The reward signal definition may be based on any of the data inputs or ML outputs described above. In an exemplary embodiment, a NIL module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module nay, update the decision-making model such that subsequently generated rankings more accurately predict optimal constraints.

Some embodiments involve the use of one or more electronic processing or computing devices. As used herein, the terms “processor” and “computer” and related terms, e.g., “processing device,” and “computing device” are not limited to just those integrated circuits referred to in the art as a computer, but broadly refers to a processor, a processing device or system, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processor (DSP), an application specific integrated circuit (ASIC), and other programmable circuits or processing devices capable of executing the functions described herein, and these terms are used interchangeably herein. These processing devices are generally “configured” to execute functions by programming or being programmed, or by the provisioning of instructions for execution. The above examples are not intended to limit in any way the definition or meaning of the terms processor, processing device, and related terms.

The various aspects illustrated by logical blocks, modules, circuits, processes, algorithms, and algorithm steps described above may be implemented as electronic hardware, software, or combinations of both. Certain disclosed components, blocks, modules, circuits, and steps are described in terms of their functionality, illustrating the interchangeability of their implementation in electronic hardware or software. The implementation of such functionality varies among different applications given varying system architectures and design constraints. Although such implementations may vary from application to application, they do not constitute a departure from the scope of this disclosure.

Aspects of embodiments implemented in software may be implemented in program code, application software, application programming interfaces (APIs), firmware, middleware, microcode, hardware description languages (HDLs), or any combination thereof. A code segment or machine-executable instruction may represent a procedure, a function, a subprogram, a program, a routine, a subroutine, a module, a software package, a class, or any combination of instructions, data structures, or program statements. A code segment may be coupled to, or integrated with, another code segment or an electronic hardware by passing or receiving information, data, arguments, parameters, memory contents, or memory locations. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the disclosed functions may be embodied, or stored, as one or more instructions or code on or in memory. In the embodiments described herein, memory includes non-transitory computer-readable media, which may include, but is not limited to, media such as flash memory, a random-access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROM, DVD, and any other digital source such as a network, a server, cloud system, or the Internet, as well as yet to be developed digital means, with the sole exception being a transitory propagating signal. The methods described herein may be embodied as executable instructions, e.g., “software” and “firmware,” in a non-transitory computer-readable medium. As used herein, the terms “software” and “firmware” are interchangeable and include any computer program stored in memory for execution by personal computers, workstations, clients, and servers. Such instructions, when executed by a processor, configure the processor to perform at least a portion of the disclosed methods.

As used herein, an element or step recited in the singular and proceeded with the word “a” or “an” should be understood as not excluding plural elements or steps unless such exclusion is explicitly recited. Furthermore, references to “one embodiment” of the disclosure or an “exemplary” or “example” embodiment are not intended to be interpreted as excluding the existence of additional embodiments that also incorporate the recited features. Likewise, limitations associated with “one embodiment” or “an embodiment” should not be interpreted as limiting to all embodiments unless explicitly recited.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein, including the implementation or utilization of components of the systems or steps independently and separately from other described components or steps. Therefore, it is manifestly intended that embodiments described herein be limited only by the claims.

TABLE 1
Listing of Sequences

| SEQ ID NO | Description |
|---|---|
| 1 | IRC7 - Saccharomyces cerevisiae S288C (NCBI Ref. No. |
| | NC_001138.5) |
| 2 | IRC7 - Saccharomyces cerevisiae S288C (NCBI Ref. No. |
| | NP_116714.1) |
| 3 | ADH4 - Saccharomyces cerevisiae S288C (NCBI Ref. No. |
| | NC_001139.9) |
| 4 | ADH4 - Saccharomyces cerevisiae S288C (NCBI Ref. No. |
| | NP_011258.2) |
| 5 | Target Sequence in IRC7 |
| 6 | Target Sequence in IRC7 |
| 7 | Target Sequence in IRC7 |
| 8 | Target Sequence in IRC7 |
| 9 | Target Sequence in IRC7 |
| 10 | Target Sequence in IRC7 |
| 11 | Target Sequence in IRC7 |
| 12 | Target Sequence in IRC7 |
| 13 | Target Sequence in IRC7 |
| 14 | Target Sequence in IRC7 |
| 15 | Target Sequence in IRC7 |
| 16 | Target Sequence in IRC7 |
| 17 | Target Sequence in IRC7 |
| 18 | Target Sequence in IRC7 |

What is claimed is:

1. A method of selecting an engineered organism with a desired protein expression, the method comprising:
receiving a request for a desired composition profile produced from a mixture;
analyzing the mixture to determine a respective quantity of a plurality of different compounds in the mixture;
selecting a gene of a yeast to interact with one or more of the compounds;
creating a plurality of gene editions based on the gene;
simulating expression of the plurality of gene editions to determine impact on the gene and a plurality of other genes;
selecting, from the plurality of gene editions, a plurality of designated gene editions that satisfy at least one predetermined criteria;
simulating a reaction with the mixture and the yeast having individual ones of the plurality of designated gene editions, wherein the simulating includes a plurality of hours, the mixture, and the yeast having the individual ones of the plurality of designated gene editions;
determining an expected final composition after a plurality of the simulated reactions;
correlating data on a plurality of attributes of the expected final compositions to the desired final composition profile;
selecting one or more expected final compositions that most closely match the desired composition profile; and
providing related gene edition data that produced the selected one or more expected final compositions.

2. The method of claim 1, wherein the mixture is feedstock for a synthetic biology production.

3. The method of claim 2, wherein the plurality of attributes includes one or more of hoppy, fruity, sulfury, bitter, floral, citrus, green, spicy, and/or sweet.

4. The method of claim 1, wherein the simulating the expression of the plurality of gene editions comprises:
inferring, based upon a tree-based machine learning algorithm, an initial metabolite precursor of n number of initial yeast cells.

5. The method of claim 4, wherein the tree-based machine learning algorithm includes a random forest regression model trained to predict an expression level of the gene based on corresponding expression levels of all other genes in the data set.

6. The method of claim 5, wherein the tree-based machine learning algorithm is constructed by recursively splitting data into smaller subsets based on expression levels of a randomly selected subset of genes until a predetermined criteria is satisfied.

7. The method of claim 6, further comprising analyzing gene interactions to determine a combined effect including one or more of a synergistic interaction, complementary interaction, and/or modifier interaction.

8. The method of claim 7, further comprising executing a regularized linear model to determine influence of perturbations on gene expression, wherein the model predicts, based on at least one combined effect of guide molecules and/or initial regulatory network granulin precursors, expression levels of the gene and the all other genes.

9. The method of claim 1, further comprising validating the selection of the engineered organism.

10. The method of claim 9, wherein validating the selection of the engineered organism comprises measuring the expression of one or more genes of the engineered organism in a fermenting liquid or a fermented product.

11. The method of claim 9, further comprising repeating the measuring the expression of one or more genes of the engineered organism in a fermenting liquid.

12. The method of claim 9, wherein measuring gene expression can comprise using high-density expression array, DNA microarray, polymerase chain reaction (PCR), reverse transcriptase PCR (RT-PCR), real-time quantitative reverse transcription PCR (qRT-PCR), serial analysis of gene expression (SAGE), spotted cDNA arrays, GeneChip, spotted oligo arrays, bead arrays, RNA Seq, tiling array, northern blotting, hybridization microarray, in situ hybridization, whole-exome sequencing, whole-genome sequencing, liquid biopsy, next-generation sequencing, or any combination thereof.

13. The method of claim 9, wherein measuring reaction data comprises analyzing the chemical composition of fermenting liquid or fermented product.

14. The method of claim 9, wherein measuring reaction data comprises analyzing the flavor composition of fermenting liquid or fermented product.

15. The method of claim 9, wherein validating the selection of the engineered organism comprises building a network of gene regulation data.

16. The method of claim 15, further comprising identifying gene regulation data associated with a desired flavor profile.

17. The method of claim 1, further comprising predicting the flavor profile based on chemical composition data.

18. The method of claim 1, wherein chemical composition data, gene expression data, and/or sensory data predicts the flavor profile of fermenting liquid or fermented product.

19. A system for selecting an engineered organism with a desired protein expression, the system comprising:
at least one memory storing instructions; and
at least one processor communicatively coupled to the at least one memory and configured to perform operations comprising:
receiving a request for a desired composition profile produced from a mixture;
analyzing the mixture to determine a respective quantity of a plurality of different compounds in the mixture;
selecting a gene of a yeast to interact with one or more of the compounds;
creating a plurality of gene editions based on the gene;
simulating expression of the plurality of gene editions to determine impact on the gene and a plurality of other genes;
selecting, from the plurality of gene editions, a plurality of designated gene editions that satisfy at least one predetermined criteria;
simulating a reaction with the mixture and the yeast having individual ones of the plurality of designated gene editions, wherein the simulating includes a plurality of hours, the mixture, and the yeast having the individual ones of the plurality of designated gene editions;
determining an expected final composition after a plurality of the simulated reactions;
correlating data on a plurality of attributes of the expected final compositions to the desired final composition profile;
selecting one or more expected final compositions that most closely match the desired composition profile; and
providing related gene edition data that produced the selected one or more expected final compositions.

20. A non-transitory computer-readable media comprising instructions stored thereon, which, when executed by at least one processor of at least one computing device for selecting an engineered organism with a desired protein expression, cause the at least one computing device to perform operations comprising:
receiving a request for a desired composition profile produced from a mixture;
analyzing the mixture to determine a respective quantity of a plurality of different compounds in the mixture;
selecting a gene of a yeast to interact with one or more of the compounds;
creating a plurality of gene editions based on the gene;
simulating expression of the plurality of gene editions to determine impact on the gene and a plurality of other genes;
selecting, from the plurality of gene editions, a plurality of designated gene editions that satisfy at least one predetermined criteria;
simulating a reaction with the mixture and the yeast having individual ones of the plurality of designated gene editions, wherein the simulating includes a plurality of hours, the mixture, and the yeast having the individual ones of the plurality of designated gene editions;
determining an expected final composition after a plurality of the simulated reactions;
correlating data on a plurality of attributes of the expected final compositions to the desired final composition profile;
selecting one or more expected final compositions that most closely match the desired composition profile; and
providing related gene edition data that produced the selected one or more expected final compositions.


SEQUENCE LISTING (18 sequences)

SEQ ID NO: 1 - DNA, 1023 bp, Saccharomyces cerevisiae
  source 1..1023: mol_type = genomic DNA
atgattgatcgtaccgagttatcgaagtttggtattactacgcaactgtctgttattggacgtaatccagatgaacaaagtggctttgttaatccacctttgtataaggggtcaaccatcattcttaaaaaacttagtgatttagaacaaaggaaaggaagattttacgggacagcaggttctccaactattgacaatttagaaaatgcctggacgcatttaaccggcggtgctgggacagtgctatcagcttctgggcttggttctatctctttggcgctattggccctttcgaaagctggtgatcatatcttgatgactgatagtgtctacgtgccaacacgtatgctatgtgatggtttattggccaagttcggtgttgaaacggattattatgacccatcaatagggaaggatatagaaaaactagttaagccaaatacaaccgtcattttcctcgaaagcccgggttctgggaccatggaagtacaggatattccagctttggtctctgttgccaaaaagcatgggataaagacaattctagacaacacatgggcaacgccactcttttttgatgctcatgcgcatggtatcgatatttcggtagaagctgggacaaaatatttgggtggtcattcagatcttcttattggtctggcctccgcaaatgaagaatgttggccgctattacggtcaacttatgatgcaatggcaatgttaccaggtgccgaggactgtcaattagcattgcgaggaatgcgtacattgcacttaagattgaaagaggtagaaagaaaagccctggatttggctgcttggctcggaaatcgagatgaggttgaaaaagtgcttcaccccgcctttgaagattgtcccggacatgaatactgggttcgtgactacaaaggttcttcaggcttattttccattgtccttaaaaatgggttcacaagagctggtctggagaaaatggtagaagggatgaaagttttgcaattgggattttcatggggtggctag

SEQ ID NO: 2 - protein, 340 aa, Saccharomyces cerevisiae
MIDRTELSKFGITTQLSVIGRNPDEQSGFVNPPLYKGSTIILKKLSDLEQRKGRFYGTAGSPTIDNLENAWTHLTGGAGTVLSASGLGSISLALLALSKAGDHILMTDSVYVPTRMLCDGLLAKFGVETDYYDPSIGKDIEKLVKPNTTVIFLESPGSGTMEVQDIPALVSVAKKHGIKTILDNTWATPLFFDAHAHGIDISVEAGTKYLGGHSDLLIGLASANEECWPLLRSTYDAMAMLPGAEDCQLALRGMRTLHLRLKEVERKALDLAAWLGNRDEVEKVLHPAFEDCPGHEYWVRDYKGSSGLFSIVLKNGFTRAGLEKMVEGMKVLQLGFSWGG

SEQ ID NO: 3 - DNA, 1149 bp, Saccharomyces cerevisiae
  source 1..1149: mol_type = genomic DNA
atgtcttccgttactgggttttacattccaccaatctctttctttggtgaaggtgctttagaagaaaccgctgattacatcaaaaacaaggattacaaaaaggctttgatcgttactgatcctggtattgcagctattggtctctccggtagagtccaaaagatgttggaagaacgtgacttaaacgttgctatctatgacaaaactcaaccaaacccaaatattgccaatgtcacagctggtttgaaggttttgaaggaacaaaactctgaaattgttgtttccattggtggtggttctgctcacgacaatgctaaggccattgctttattggctactaacggtggggaaatcggagactatgaaggtgtcaatcaatctaagaaggctgctttaccactatttgccatcaacactactgctggtactgcttccgaaatgaccagattcactattatctctaatgaagaaaagaaaatcaagatggctatcattgacaacaacgtcactccagctgttgctgtcaacgatccatctaccatgtttggtttgccacctgctttgactgctgctactggtctagatgctttgactcactgtatcgaagcttatgtttccaccgcctctaacccaatcaccgatgcctgtgctttgaagggtattgatttgatcaatgaaagcttagtcgctgcatacaaagacggtaaagacaagaaggccagaactgacatgtgttacgctgaatacttggcaggtatggctttcaacaatgcttctctaggttatgttcatgcccttgctcatcaacttggtggtttctaccacttgcctcatggtgtttgtaacgctgtcttgttgcctcatgttcaagaggccaacatgcaatgtccaaaggccaagaagagattaggtgaaattgctttgcatttcggtgcttctcaagaagatccagaagaaaccatcaaggctttgcacgttttaaacagaaccatgaacattccaagaaacttgaaagaattaggtgttaaaaccgaagattttgaaattttggctgaacacgccatgcatgatgcctgccatttgactaacccagttcaattcaccaaagaacaagtggttgccattatcaagaaagcctatgaatattaa

SEQ ID NO: 4 - protein, 382 aa, Saccharomyces cerevisiae
MSSVTGFYIPPISFFGEGALEETADYIKNKDYKKALIVTDPGIAAIGLSGRVQKMLEERDLNVAIYDKTQPNPNIANVTAGLKVLKEQNSEIVVSIGGGSAHDNAKAIALLATNGGEIGDYEGVNQSKKAALPLFAINTTAGTASEMTRFTIISNEEKKIKMAIIDNNVTPAVAVNDPSTMFGLPPALTAATGLDALTHCIEAYVSTASNPITDACALKGIDLINESLVAAYKDGKDKKARTDMCYAEYLAGMAFNNASLGYVHALAHQLGGFYHLPHGVCNAVLLPHVQEANMQCPKAKKRLGEIALHFGASQEDPEETIKALHVLNRTMNIPRNLKELGVKTEDFEILAEHAMHDACHLTNPVQFTKEQVVAIIKKAYEY

SEQ ID NO: 5 - DNA, 23 bp, Saccharomyces cerevisiae
  source 1..23: mol_type = genomic DNA
tgataaaaatatagaagacaagg

SEQ ID NO: 6 - DNA, 23 bp, Saccharomyces cerevisiae
  source 1..23: mol_type = genomic DNA
cgttaagtattttttcattgtgg

SEQ ID NO: 7 - DNA, 23 bp, Saccharomyces cerevisiae
  source 1..23: mol_type = genomic DNA
aagaaagactgctctagtgttgg

SEQ ID NO: 8 - DNA, 23 bp, Saccharomyces cerevisiae
  source 1..23: mol_type = genomic DNA
gtataatcaactcaattgtaagg

SEQ ID NO: 9 - DNA, 23 bp, Saccharomyces cerevisiae
  source 1..23: mol_type = genomic DNA
cactttatgaatgttagatacgg

SEQ ID NO: 10 - DNA, 23 bp, Saccharomyces cerevisiae
  source 1..23: mol_type = genomic DNA
agaaagactgctctagtgttggg

SEQ ID NO: 11 - DNA, 23 bp, Saccharomyces cerevisiae
  source 1..23: mol_type = genomic DNA
tgtcttctatatttttatcaagg

SEQ ID NO: 12 - DNA, 23 bp, Saccharomyces cerevisiae
  source 1..23: mol_type = genomic DNA
tttagcactgatgagcaaattgg

SEQ ID NO: 13 - DNA, 23 bp, Saccharomyces cerevisiae
  source 1..23: mol_type = genomic DNA
ttacaattgagttgattatacgg

SEQ ID NO: 14 - DNA, 23 bp, Saccharomyces cerevisiae
  source 1..23: mol_type = genomic DNA
gtgaatattttggtttttgctgg

SEQ ID NO: 15 - DNA, 23 bp, Saccharomyces cerevisiae
  source 1..23: mol_type = genomic DNA
aatcattgtagtgaatattttgg

SEQ ID NO: 16 - DNA, 23 bp, Saccharomyces cerevisiae
  source 1..23: mol_type = genomic DNA
ttggcactttcaacattatttgg

SEQ ID NO: 17 - DNA, 23 bp, Saccharomyces cerevisiae
  source 1..23: mol_type = genomic DNA
aaaaatatactcaaaacaagagg

SEQ ID NO: 18 - DNA, 23 bp, Saccharomyces cerevisiae
  source 1..23: mol_type = genomic DNA
tcaacattatttggatgctacgg